(19) United States
(12) Patent Application Publication
(10) Pub. No.: US 2025/0171456 A1
(43) Pub. Date: May 29, 2025

(54) PSYCHEDELIC COMPOUNDS AND THEIR THERAPEUTIC USES

(71) Applicant: CAAMTECH, INC., Issaquah, WA (US)

(72) Inventor: Robert DISCORDIA, Denver, NC (US)

(21) Appl. No.: 18/834,458

(22) PCT Filed: Feb. 1, 2023
(86) PCT No.: PCT/US2023/061744

§ 371 Date: Jul. 30, 2024

Related U.S. Application Data
(60) Provisional application No. 63/305,309, filed on Feb. 1, 2022.
(60) Provisional application No. 63/305,312, filed on Feb. 1, 2022.
(60) Provisional application No. 63/305,314, filed on Feb. 1, 2022.
(60) Provisional application No. 63/305,315, filed on Feb. 1, 2022.
(60) Provisional application No. 63/375,459, filed on Sep. 13, 2022.

(51) Int. Cl. C07D 487/14; A61K 31/396; A61K 31/397; A61K 31/4025; A61K 31/404; A61K 31/437; A61K 31/4525; A61K 31/454; A61K 31/55; A61K 45/06; C07D 401/06; C07D 403/06; C07D 405/06; C07D 471/14; C07D 487/18

(52) U.S. Cl. CPC C07D 487/14; A61K 31/396; A61K 31/397; A61K 31/4025; A61K 31/404; A61K 31/437; A61K 31/4525; A61K 31/454; A61K 31/55; A61K 45/06; C07D 401/06; C07D 403/06; C07D 405/06; C07D 471/14; C07D 487/18

(57) ABSTRACT

The disclosure relates to compounds of formulae (I), (II), (III), and (IV): The disclosure relates to compositions comprising, consisting essentially of, or consisting of a compound of formulae (I), (II), (III), or (IV) and an excipient. The disclosure also relates to pharmaceutical compositions comprising a therapeutically effective amount of a compound of formulae (I), (II), (III), or (IV) where the excipient is a pharmaceutically acceptable carrier. The disclosure further relates to therapeutic uses of compounds of formulae (I), (II), (III), or (IV).

### CROSS REFERENCE TO RELATED APPLICATIONS

This application claims priority to U.S. Provisional Application No. 63/305,309, filed on Feb. 1, 2022; U.S. Provisional Application No. 63/305,312, filed on Feb. 1, 2022; U.S. Provisional Application No. 63/305,314, filed on Feb. 1, 2022; U.S. Provisional Application No. 63/305,315, filed on Feb. 1, 2022; and U.S. Provisional Application No. 63/375,459, filed on Sep. 13, 2022; the disclosures of which are incorporated by reference.

### TECHNICAL FIELD

This disclosure relates to tryptamine compounds, compositions, and pharmaceutical compositions containing them as well as their use in treating various diseases.

This disclosure further relates to phenethylamine compounds, compositions, and pharmaceutical compositions containing them as well as their use in treating various diseases.

### BACKGROUND

Ibogaine (CAS 83-74-9) is a psychedelic tryptamine alkaloid first isolated in 1901 from the root bark of the Tabernanthe iboga shrub (also called iboga) of Central Africa and the root bark of a shrub in the genus Tabernaemontana found in the Congo (Dybowski, 1901; Haller, 1901). Since that time, ibogaine has been isolated from several plant species particularly those in the family Apocynaceae to which T. iboga belongs. Plants containing ibogaine as well as extracts and preparations made from them include many other active and inactive molecules including several different ibogaine derivatives. Ibogaine exhibits relatively low potency at target sites. Studies in rats suggest high doses may cause adverse effects. In 1988, Dzoljic et al. were the first to publish on the ability of ibogaine to relieve withdrawal from narcotics addiction (Dzolkic, 1988). Maisonneuve et al. elucidated the pharmacological interactions between ibogaine and morphine in 1991 (Maisonneuve, 1991). After this, several other researchers showed ibogaine's ability to reduce or interrupt the self-administration of opiates in rats and mice and alter their behaviors (Glick, 1991; Cappendijk, 1993; Broderick, 1985; Broderick, 1994; Sershen, 1994). Additional study results showed ibogaine was more effective in multiple administrations over time than from a single dose (Glick, 1991; Cappendijk, 1993). Recent review papers and meta-analyses have concluded that ibogaine is effective for treating substance addiction and warrants further investigation (Schenberg, 2014; Winkelman, 2014; Belgers, 2016). Several current studies have found ibogaine effective for treating opioid addiction (Noller, 2018, Mash, 2018; Brown, 2018; Davis, 2017).

Psilocybin is a breakthrough drug that has received FDA approval for therapeutic applications. Psilocybin is one of several naturally occurring psychoactive tryptamines found in “magic” mushrooms. When consumed by humans, psilocybin serves as a prodrug of psilocin. Psilocin is a potent serotonin 2a-agonist, which is responsible for its psychoactive properties (Dinis-Oliveira, 2017; Nichols, 2012). Upon digestion, intestinal alkaline phosphate dephosphorylates psilocybin to generate psilocin (Horita and Weber, 1961). Psychoactive tryptamines like psilocin have garnered significant interest recently because of their potential for treating mood disorders, including depression, anxiety, addiction, and post-traumatic stress disorder (PTSD) (Johnson & Griffiths, 2017; Carhart-Harris & Goodwin, 2017). Altering the chemical structure within this class of compounds can dramatically influence the potency and action of the drugs. Psilocybin is converted to psilocin by an enzyme-catalyzed hydrolysis reaction. In humans it was found that after oral administration, psilocin is detectable in significant amounts in the plasma within 20-40 minutes (Passie, 2002). Psilocybin is more soluble in water than psilocin (Ballesteros, 2006). Therefore, psilocin is more easily absorbed from the rat jejunum and colon gastrointestinal track, which suggests greater central nervous system bioavailability (Eivindvik, 1989). Therefore, there is a need for rationally designed analogs of already psychoactive naturally occurring substances to enhance or throttle back activity, reduce side-effects, increase bioavailability and/or other pharmacokinetic and therapeutic properties for treating human conditions.

The compound 3,4-methylenedioxymethamphetamine (MDMA) is a serotonergic drug that enhances serotonergic neurotransmission via release of 5-HT through the serotonin transporter (SERT). MDMA is also known to trigger oxytocin release which may contribute to its effects to increase trust, prosociality, and enhanced empathy. Although psychedelics, such as MDMA, have significant potential for treating many mood disorders, such treatment options often have side-effects which can be generally categorized as “dysphoria.” This dysphoric or unpleasant subjective effects include negative thoughts, rumination, anxiety, panic, paranoia, loss of trust towards other people and perceived loss of control, depending on the dose of the psychedelic drug used, the personality traits of the person consuming it (i.e., ‘set’), the environment in which it is consumed (i.e., ‘setting’), and other factors yet to be determined. Acute negative psychological effects are considered the main risk of psychedelic substance use in humans. There is an unmet need for mitigating these acute negative psychological effects when administering psychedelic drugs to human subjects. There is an unmet need for methods which are capable of reducing bad drug effects while enhancing good drug effects to optimize a psychedelic experience. Altering the chemical structure within this class of compounds can dramatically influence the potency and action of the drugs.

### SUMMARY OF THE DISCLOSURE

The disclosure relates to a compound of formula (I):

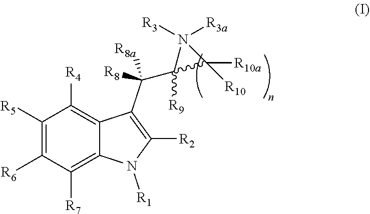

wherein:
   R₁ is selected from hydrogen, optionally substituted C₁-C₆ alkyl, optionally substituted C₂-C₆ alkenyl, optionally substituted aryl, —OR₁₁, —C(O)R₁₁, —C(O)OR₁₁, —SO₂R₁₁, and —C(O)NR₁₁R₁₂;
   R₂, R₄, R₅, R₆, R₇, R₈, R₈ₐ, R₉, R₁₀, and R₁₀ₐ are for each occurrence independently selected from hydrogen, optionally substituted C₁-C₆ alkyl, optionally substituted C₂-C₆ alkenyl, optionally substituted C₁-C₆-heteroalkyl, optionally substituted heteroaryl, optionally substituted aryl, halogen, hydroxy, CN, —OR₁₁, —OC(O)R₁₁, —OC(O)OR₁₁, —OSO₂R₁₁, —OC(O)NR₁₁R₁₂, —OP(O)(OH)₂, and —OP(O)(OH)OP(O)(OH)₂;
   R₃ is selected from hydrogen, optionally substituted C₁-C₆ alkyl, optionally substituted C₂-C₆ alkenyl, optionally substituted aryl, and —SO₂R₁₁;
   R₃ₐ is selected from an electron pair, hydrogen, optionally substituted C₁-C₆ alkyl, optionally substituted C₂-C₆ alkenyl, and optionally substituted aryl, wherein when R₃ₐ is not an electron pair the compound of formula (I) is a quaternary amine cation with a pharmaceutically acceptable anion, X⁻;
   R₁₁ and R₁₂ are for each occurrence independently selected from hydrogen, optionally substituted C₁-C₆ alkyl, optionally substituted C₂-C₆ alkenyl, and optionally substituted aryl; and
   n is an integer defining the variable number of ring carbons carrying R₁₀ and R₁₀ₐ and is selected from 1 to 4.

The disclosure further relates to a compound of formula (11):

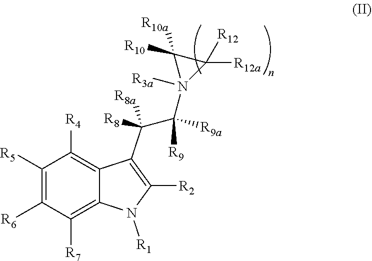

wherein:
   R₁ is selected from hydrogen, optionally substituted C₁-C₆ alkyl, optionally substituted C₂-C₆ alkenyl, optionally substituted aryl, —OR₁₁, —C(O)R₁₁, —C(O)OR₁₁, —SO₂R₁₁, and —C(O)NR₁₁R₁₃;
   R₂, R₄, R₅, R₆, R₇, R₈, R₈ₐ, R₉, R₉ₐ, R₁₀, R₁₀ₐ, R₁₂, and R₁₂ₐ are for each occurrence independently selected from hydrogen, optionally substituted C₁-C₆ alkyl, optionally substituted C₂-C₆ alkenyl, optionally substituted C₁-C₆-heteroalkyl, optionally substituted heteroaryl, optionally substituted aryl, halogen, hydroxy, CN, —OR₁₁, —OC(O)R₁₁, —OC(O)OR₁₁, —OSO₂R₁₁, —OC(O)NR₁₁R₁₃, —OP(O)(OH)₂, and —OP(O)(OH)OP(O)(OH)₂;
   R₃ₐ is selected from an electron pair, hydrogen, optionally substituted C₁-C₆ alkyl, optionally substituted C₂-C₆ alkenyl, and optionally substituted aryl, wherein when R₃ₐ is not an electron pair the compound of formula (II) is a quaternary amine cation with a pharmaceutically acceptable anion, X⁻;
   R₁₁ and R₁₃ are for each occurrence independently selected from hydrogen, optionally substituted C₁-C₆ alkyl, optionally substituted C₂-C₆ alkenyl, and optionally substituted aryl; and
   n is an integer defining the variable number of ring carbons carrying R₁₂ and R₁₂ₐ and is selected from 1 to 4.

The disclosure further relates to a compound of formula (III):

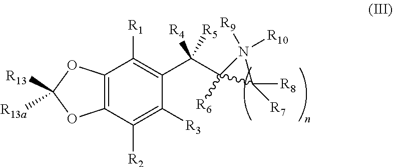

wherein:
   R₁, R₂, and R₃ are independently selected from hydrogen, optionally substituted C₁-C₆ alkyl, optionally substituted C₂-C₆ alkenyl, optionally substituted aryl, halogen, —OR₁₁, —C(O)R₁₁, —C(O)OR₁₁, —OC(O)R₁₁, C(O)NR₁₁R₁₂, CN, C(NR₁₂)R₁₁, C(NOR₁₂)OR₁₁, —SO₂R₁₁, and —C(O)NR₁₁R₁₂;
   R₄, R₅, R₆, R₇, and R₈ are for each occurrence independently selected from hydrogen, optionally substituted C₁-C₆ alkyl, optionally substituted C₂-C₆ alkenyl, optionally substituted C₁-C₆ heteroalkyl, optionally substituted heteroaryl, optionally substituted aryl, halogen, hydroxy, —OR₁₁, —OC(O)R₁₁, —OC(O)OR₁₁, —OSO₂R₁₁, and —OC(O)NR₁₁R₁₂;
   R₉ is selected from hydrogen, optionally substituted C₁-C₆ alkyl, optionally substituted C₂-C₆ alkenyl, optionally substituted aryl, and —SO₂R₁₁;
   R₁₀ is selected from an electron pair, hydrogen, optionally substituted C₁-C₆ alkyl, optionally substituted C₂-C₆ alkenyl, and optionally substituted aryl, wherein when R₁₀ is not an electron pair the compound of formula (III) is a quaternary amine cation with a pharmaceutically acceptable anion, X⁻;
   R₁₁ and R₁₂ are for each occurrence independently selected from hydrogen, optionally substituted C₁-C₆ alkyl, optionally substituted C₂-C₆ alkenyl, and optionally substituted aryl;
   R₁₃ and R₁₃ₐ are for each occurrence independently selected from hydrogen, optionally substituted C₁-C₆ alkyl, optionally substituted C₂-C₆ alkenyl, and optionally substituted aryl, or R₁₃ and
   R₁₃ₐ are taken together with the carbon to which they are bound to form a 3- to 6-membered cycloalkyl; and
   n is an integer defining the variable number of ring carbons carrying R₇ and R₈ and is selected from 1 to 4.

The disclosure further relates to a compound of formula (IV):

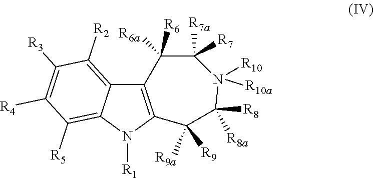

wherein:
   R₁ is selected from hydrogen, optionally substituted C₁-C₆ alkyl, optionally substituted C₂-C₆ alkenyl, optionally substituted aryl, —OR₁₁, —C(O)R₁₁, —C(O)OR₁₁, —SO₂R₁₁, and —C(O)NR₁₁R₁₂;
   R₂, R₃, R₄, R₅, R₆, R₆ₐ, R₇, R₇ₐ, R₈, R₈ₐ, R₉, and R₉ₐ are for each occurrence independently selected from hydrogen, optionally substituted C₁-C₆ alkyl, optionally substituted C₂-C₆ alkenyl, optionally substituted C₁-C₆-heteroalkyl, optionally substituted heteroaryl, optionally substituted aryl, halogen, hydroxy, —OR₁₁, —OC(O)R₁₁, —OC(O)OR₁₁, —OSO₂R₁₁, and —OC(O)NR₁₁R₁₂;
   R₁₀ is selected from an electron pair, hydrogen, optionally substituted C₁-C₆ alkyl, optionally substituted C₂-C₆ alkenyl, and optionally substituted aryl, wherein when R₁₀ is not an electron pair the compound of formula (IV) is a quaternary amine cation with a pharmaceutically acceptable anion, X⁻;
   R₁₀ₐ is taken together with one of R₈, R₈ₐ, R₉, or R₉ₐ to form optionally substituted 3- to 6-membered heterocyclic ring; and
   R₁₁ and R₁₂ are for each occurrence independently selected from hydrogen, optionally substituted C₁-C₆ alkyl, optionally substituted C₂-C₆ alkenyl, and optionally substituted aryl.

The disclosure relates to compositions comprising, consisting essentially of, or consisting of a compound of formulae (I), (II), (III), or (IV) and an excipient. The disclosure also relates pharmaceutical compositions comprising a therapeutically effective amount of a compound of formulae (I), (II), (III), or (IV), wherein the excipient is a pharmaceutically acceptable carrier. The disclosure further relates to a method of preventing or treating a psychological disorder comprising the step of administering to a subject in need thereof a therapeutically effective amount of a compound of formulae (I), (II), (III), or (IV), or a pharmaceutical composition according to this disclosure.

The disclosure also relates to a composition comprising, consisting essentially of, or consisting of as a first active component: a compound of formulae (I), (II), (III), or (IV); and as a second active component selected from (a) a serotonergic drug, (b) a purified psilocybin derivative, (c) a purified cannabinoid, (d) a purified terpene, (e) an adrenergic drug, (f) a dopaminergic drug, (g) a monoamine oxidase inhibitor, (h) a purified erinacine, and (i) a purified hericenone; and a pharmaceutically acceptable excipient.

The disclosure further relates to methods of preventing or treating a physical and/or psychological disorders comprising the step of administering to a subject in need thereof an effective amount of a compound of formulae (I), (II), (III), or (IV), or a composition (e.g., a pharmaceutically-acceptable composition) comprising a compound of formulae (I), (II), (III), or (IV), according to this disclosure.

The disclosure further relates to methods of treating and/or preventing a substance use disorder comprising the step of administering to a subject in need thereof a therapeutically effective amount of a compound of formulae (I), (II), (III), or (IV), or a composition (e.g., a pharmaceutically-acceptable composition) comprising a compound of formulae (I), (II), (III), or (IV), according to the disclosure.

The disclosure also relates to methods of preventing or treating inflammation and/or pain comprising the step of administering to a subject in need thereof a therapeutically effective amount of a compound of formulae (I), (II), (III), or (IV), and to administering a pharmaceutical composition or a composition according to the disclosure.

The disclosure also relates to methods of preventing or treating inflammation and/or pain, preventing or treating a neurological disorder, modulating activity of a mitogen-activated protein kinase (MAPK), modulating neurogenesis, or modulating neurite outgrowth comprising the step of administering to a subject in need thereof a therapeutically effective amount of a compound of formulae (I), (II), (III), or (IV), and to administering a pharmaceutical composition or a composition according to the disclosure.

The disclosure also relates to methods of preventing or treating sexual health disorders including, but not limited to, hypoactive sexual desire disorder, hyperactive sexual desire disorder, orgasmic disorder, arousal disorder, vaginismus, and dyspareunia, comprising the step of administering to a subject in need thereof a therapeutically effective amount of a compound of formulae (I), (II), (III), or (IV), and to administering a pharmaceutical composition or a composition according to the disclosure. In some embodiments, the disorder is a male sexual dysfunction disorder. In some embodiments, the disorder is a female sexual dysfunction disorder.

The disclosure also relates to methods of preventing or treating women's health disorders including, but not limited to, menstrual cramping, dysmenorrhea, post-hysterectomy pain, vaginal or vulvar vestibule mucosa disorder, vaginal atrophy, or vulvar vestibulitis, comprising the step of administering to a subject in need thereof a therapeutically effective amount of a compound of formulae (I), (II), (III), or (IV), and to administering a pharmaceutical composition or a composition according to the disclosure.

As used herein, the term “a subject in need thereof” refers to a person requiring a composition to treat a particular disease or condition (e.g., inflammation, pain, a psychological disorder, modulating activity at a receptor, etc.). In one embodiment, the “subject in need thereof” may be identified by analyzing, diagnosing, and/or determining whether the person (or subject) requires the composition for treatment of a particular disease or condition. In one embodiment, identifying a person in need of treatment comprises diagnosing a person with a medical condition, e.g., a neurological disorder, a chemical imbalance, a hereditary condition, etc. In one embodiment, identifying a person in need of treatment comprises performing a psychiatric evaluation. In one embodiment, identifying a person in need of treatment comprises performing a blood test. In one embodiment, identifying a person in need of treatment comprises determining whether a person has a compulsive disorder. In one embodiment, identifying a person in need of treatment comprises self-identifying as having a compulsive disorder.

### DESCRIPTION OF THE FIGURES

FIG. 1 shows the ¹H NMR spectrum of (S)-3-((1-methylaziridin-2-yl)methyl)-1H-indol-4-ol.

FIG. 2 shows the ¹H NMR spectrum of Azacyclopropyl-MDMA analog.

### DETAILED DESCRIPTION

### Compounds of the Disclosure

The disclosure specifically relates to the purposeful design of novel molecular structures, so designed to impart rotational and conformational restrictions at the loci of putative binding of the subject molecules with CNS receptors. These modifications involve the “tying up” of otherwise freely rotational moieties into conformations that cause bond angles and bond lengths to be changed to afford useful improvements in biological profile over the naturally derived substances. The effects are enhancements to the pharmacological profile of the naturally derived parent molecules to be useful in psychiatric therapy for presently unmet medical needs. There is a wealth of information on the utility of certain hallucinogenic substances to possess the ability to treat heretofore hard to treat or untreatable psychological ailments such as depression, anxiety, post-traumatic stress disorder (PTSD), traumatic brain injury (TBI), chronic traumatic encephalopathy (CTE), etc.

The derivatization of psilocin, such as from a structure-activity relationship, indicates a significant diminution of affinity and efficacy at 5HT_{2A}R and other receptors in proportion to increasing steric bulk about the terminal N,N-dialkylaminoethyl side chain functionality (Klein) circled in the following chemical structure:

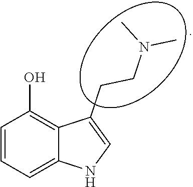

It is reasoned that the binding affinity to and fidelity for critical receptors may be modulated by conformational and/or rotational restriction about the moieties that bind the receptor sites. In addition, changes in C—N—C atomic bond lengths and nitrogen hybridization and barrier to pyramidal inversion can also be levers to adjust pharmacological properties (Padwa; Katritzky). To that end, tryptamine and serotonin analogs have been designed and the following Markush structure depicting the scope of such derivatives is shown in formula (I) below. A key feature of formula (I) is the appearance of small 3- to 6-membered ring heterocycles which change bond lengths and angles in the N,N-dimethylamino-ethyl moiety of Psilocybin/Psilocin. Formula (I) also introduces chirality at one or more of the ring carbons, which also modulates activity. Additional modulation of activity can be brought about through changes in the electronics of the indole ring through substitution with different elements.

The disclosure relates to a compound of formula (I):

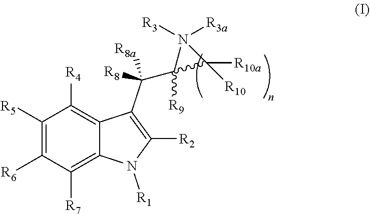

wherein:
   R₁ is selected from hydrogen, optionally substituted C₁-C₆ alkyl, optionally substituted C₂-C₆ alkenyl, optionally substituted aryl, —OR₁₁, —C(O)R₁₁, —C(O)OR₁₁, —SO₂R₁₁, and —C(O)NR₁₁R₁₂;
   R₂, R₄, R₅, R₆, R₇, R₈, R₈ₐ, R₉, R₁₀, and R₁₀ₐ are for each occurrence independently selected from hydrogen, optionally substituted C₁-C₆ alkyl, optionally substituted C₂-C₆ alkenyl, optionally substituted C₁-C₆-heteroalkyl, optionally substituted heteroaryl, optionally substituted aryl, halogen, hydroxy, CN, —OR₁₁, —OC(O)R₁₁, —OC(O)OR₁₁, —OSO₂R₁₁, —OC(O)NR₁₁R₁₂, —OP(O)(OH)₂, and —OP(O)(OH)OP(O)(OH)₂;
   R₃ is selected from hydrogen, optionally substituted C₁-C₆ alkyl, optionally substituted C₂-C₆ alkenyl, optionally substituted aryl, and —SO₂R₁₁;
   R₃ₐ is selected from an electron pair, hydrogen, optionally substituted C₁-C₆ alkyl, optionally substituted C₂-C₆ alkenyl, and optionally substituted aryl, wherein when R₃ₐ is not an electron pair the compound of formula (I) is a quaternary amine cation with a pharmaceutically acceptable anion, X⁻;
   R₁₁ and R₁₂ are for each occurrence independently selected from hydrogen, optionally substituted C₁-C₆ alkyl, optionally substituted C₂-C₆ alkenyl, and optionally substituted aryl; and
   n is an integer defining the variable number of ring carbons carrying R₁₀ and R₁₀ₐ and is selected from 1 to 4.

In formula (I), R₁ is selected from hydrogen, optionally substituted C₁-C₆ alkyl, optionally substituted C₂-C₆ alkenyl, optionally substituted aryl, —OR₁₁, —C(O)R₁₁, —C(O)OR₁₁, —SO₂R₁₁, and —C(O)NR₁₁R₁₂. R₁ may be hydrogen. R₁ may be an optionally substituted straight chain or branched C₁-C₆ alkyl, for example a straight chain C₁-C₆ alkyl, or an optionally substituted straight chain or branched C₂-C₆ alkenyl, for example allyl, 2-butenyl, etc. In some embodiments R₁ may be a straight chain or branched C₁-C₄ alkyl, for example a straight chain C₁-C₄ alkyl, or a C₂-C₄ alkenyl. R₁ may be an optionally substituted aryl. An aryl is a 6- to 14-membered aromatic ring, preferably a 6- to 10-membered aromatic ring and includes polycyclic ring systems in which two or more carbon atoms are common to adjoining rings where at least one ring is aromatic. Examples of aryl groups include, but are not limited to phenyl, naphthyl, anthracenyl, and phenantherenyl. An aryl group may be substituted with one or more C₁-C₄ alkyl or perfluoralkyl groups, C₁-C₄ hydroxyalkyl groups, hydroxyl groups, nitro groups or halo groups (e.g., F, Cl, I, or Br). An aryl group may be ortho-, meta-, and/or para-substituted, preferably para-substituted. R₁ may be —OR₁₁, —C(O)R₁₁, —C(O)OR₁₁, —SO₂R₁₁, or —C(O)NR₁₁R₁₂, wherein Ru and R₁₂ are for each occurrence independently selected from hydrogen, optionally substituted C₁-C₆ alkyl, optionally substituted C₂-C₆ alkenyl, and optionally substituted aryl.

In formula (I), R₂, R₄, R₅, R₆, R₇, R₈, R₈ₐ, R₉, R₁₀, and R₁₀ₐ are for each occurrence independently selected from hydrogen, optionally substituted C₁-C₆ alkyl, optionally substituted C₂-C₆ alkenyl, optionally substituted C₁-C₆-heteroalkyl, optionally substituted heteroaryl, optionally substituted aryl, halogen, hydroxy, CN, —OR₁₁, —OC(O)R₁₁, —OC(O)OR₁₁, —OSO₂R₁₁, —OC(O)NR₁₁R₁₂, —OP(O)(OH)₂, and —OP(O)(OH)OP(O)(OH)₂. R₂, R₄, R₅, R₆, R₇, R₈, R₈ₐ, R₉, R₁₀, and R₁₀ₐ may each independently be hydrogen. R₂, R₄, R₅, R₆, R₇, R₈, R₈ₐ, R₉, R₁₀, and R₁₀ₐ may each independently be an optionally substituted straight chain or branched C₁-C₆ alkyl, for example, a straight chain C₁-C₆ alkyl, or an optionally substituted straight chain or branched C₂-C₆ alkenyl, for example allyl, 2-butenyl, etc. In some embodiments R₂, R₄, R₅, R₆, R₇, R₈, R₈ₐ, R₉, R₁₀, and R₁₀ₐ may each independently be a straight chain or branched C₁-C₄ alkyl, for example a straight chain C₁-C₄ alkyl, or a C₂-C₄ alkenyl. R₂, R₄, R₅, R₆, R₇, R₈, R₈ₐ, R₉, R₁₀, and R₁₀ₐ may each independently be an optionally substituted C₁-C₆-heteroalkyl, including but not limited to alkoxy, alkylthio, and alkylamino. R₂, R₄, R₅, R₆, R₇, R₈, R₈ₐ, R₉, R₁₀, and R₁₀ₐ may each independently be an optionally substituted heteroaryl, including, but not limited to, furano, pyridinyl, pyrimidinyl, etc. R₂, R₄, R₅, R₆, R₇, R₈, R₈ₐ, R₉, R₁₀, and R₁₀ₐ may each independently be an optionally substituted aryl. An aryl is a 6- to 14-membered aromatic ring, preferably a 6- to 10-membered aromatic ring and includes polycyclic ring systems in which two or more carbon atoms are common to adjoining rings where at least one ring is aromatic. Examples of aryl groups include, but are not limited to phenyl, naphthyl, anthracenyl, and phenantherenyl. An aryl group may be substituted with one or more C₁-C₄ alkyl or perfluoralkyl groups, C₁-C₄ hydroxyalkyl groups, hydroxyl groups, nitro groups or halo groups (e.g., F, Cl, I, or Br). An aryl group may be ortho-, meta-, and/or para-substituted, preferably para-substituted. R₂, R₄, R₅, R₆, R₇, R₈, R₈ₐ, R₉, R₁₀, and R₁₀ₐ may each independently be a halogen. Exemplary halogens include fluorine (F), chlorine (Cl), bromine (Br), and iodine (I). R₂, R₄, R₅, R₆, R₇, R₈, R₈ₐ, R₉, R₁₀, and R₁₀ₐ may each independently be hydroxy. R₂, R₄, R₅, R₆, R₇, R₈, R₈ₐ, R₉, R₁₀, and R₁₀ₐ may each independently be CN. R₂, R₄, R₅, R₆, R₇, R₈, R₈ₐ, R₉, R₁₀, and R₁₀ₐ may each independently be —OR₁₁, —OC(O)R₁₁, —OC(O)OR₁₁, —OSO₂R₁₁, —OC(O)NR₁₁R₁₂, —OP(O)(OH)₂, or —OP(O)(OH)OP(O)(OH)₂, wherein R₁₁ and R₁₂ are for each occurrence independently selected from hydrogen, optionally substituted C₁-C₆ alkyl, optionally substituted C₂-C₆ alkenyl, and optionally substituted aryl.

In formula (I), R₃ is selected from hydrogen, optionally substituted C₁-C₆ alkyl, optionally substituted C₂-C₆ alkenyl, optionally substituted aryl, and —SO₂R₁₁. R₃ may be hydrogen. R₃ may be an optionally substituted straight chain or branched C₁-C₆ alkyl, for example a straight chain C₁-C₆ alkyl, or an optionally substituted straight chain or branched C₂-C₆ alkenyl, for example allyl, 2-butenyl, etc. In some embodiments R₃ may be a straight chain or branched C₁-C₄ alkyl, for example a straight chain C₁-C₄ alkyl, or a C₂-C₄ alkenyl. R₃ may be an optionally substituted aryl. An aryl is a 6- to 14-membered aromatic ring, preferably a 6- to 10-membered aromatic ring and includes polycyclic ring systems in which two or more carbon atoms are common to adjoining rings where at least one ring is aromatic.

Examples of aryl groups include, but are not limited to phenyl, naphthyl, anthracenyl, and phenantherenyl. An aryl group may be substituted with one or more C₁-C₄ alkyl or perfluoralkyl groups, C₁-C₄ hydroxyalkyl groups, hydroxyl groups, nitro groups or halo groups (e.g., F, Cl, I, or Br). An aryl group may be ortho-, meta-, and/or para-substituted, preferably para-substituted. R₃ may be —SO₂R₁₁, wherein R₁₁ is selected from hydrogen, optionally substituted C₁-C₆ alkyl, optionally substituted C₂-C₆ alkenyl, and optionally substituted aryl.

In formula (I), R₃ₐ is selected from an electron pair, hydrogen, optionally substituted C₁-C₆ alkyl, optionally substituted C₂-C₆ alkenyl, and optionally substituted aryl, wherein when R₃ₐ is not an electron pair the compound of formula (I) is a quaternary amine cation with a pharmaceutically acceptable anion, X⁻. R₃ₐ may be an electron pair. R₃ₐ may be hydrogen. R₃ₐ may be an optionally substituted straight chain or branched C₁-C₆ alkyl, for example a straight chain C₁-C₆ alkyl, or an optionally substituted straight chain or branched C₂-C₆ alkenyl, for example allyl, 2-butenyl, etc. In some embodiments R₃ₐ may be a straight chain or branched C₁-C₄ alkyl, for example a straight chain C₁-C₄ alkyl, or a C₂-C₄ alkenyl. R₃, may be an optionally substituted aryl. An aryl is a 6- to 14-membered aromatic ring, preferably a 6- to 10-membered aromatic ring and includes polycyclic ring systems in which two or more carbon atoms are common to adjoining rings where at least one ring is aromatic. Examples of aryl groups include, but are not limited to phenyl, naphthyl, anthracenyl, and phenantherenyl. An aryl group may be substituted with one or more C₁-C₄ alkyl or perfluoralkyl groups, C₁-C₄ hydroxyalkyl groups, hydroxyl groups, nitro groups or halo groups (e.g., F, Cl, I, or Br). An aryl group may be ortho-, meta-, and/or para-substituted, preferably para-substituted.

The anion of formula (I), X⁻, may be any pharmaceutically acceptable anion, for example, Cl⁻, I⁻, Br, ascorbate, hydrofumarate, fumarate, maleate, and the like.

In formula (I), R₁₁ and R₁₂ are for each occurrence independently selected from hydrogen, optionally substituted C₁-C₆ alkyl, optionally substituted C₂-C₆ alkenyl, and optionally substituted aryl. R₁₁ and R₁₂ may each independently be hydrogen. R₁₁ and R₁₂ may each independently be an optionally substituted straight chain or branched C₁-C₆ alkyl, for example a straight chain C₁-C₆ alkyl, or an optionally substituted straight chain or branched C₂-C₆ alkenyl, for example allyl, 2-butenyl, etc. In some embodiments R₁₁ and R₁₂ may each independently be a straight chain or branched C₁-C₄ alkyl, for example a straight chain C₁-C₄ alkyl, or a C₂-C₄ alkenyl. R₁₁ and R₁₂ may each independently be an optionally substituted aryl. An aryl is a 6- to 14-membered aromatic ring, preferably a 6- to 10-membered aromatic ring and includes polycyclic ring systems in which two or more carbon atoms are common to adjoining rings where at least one ring is aromatic. Examples of aryl groups include, but are not limited to phenyl, naphthyl, anthracenyl, and phenantherenyl. An aryl group may be substituted with one or more C₁-C₄ alkyl or perfluoralkyl groups, C₁-C₄ hydroxyalkyl groups, hydroxyl groups, nitro groups or halo groups (e.g., F, Cl, I, or Br). An aryl group may be ortho-, meta-, and/or para-substituted, preferably para-substituted.

In formula (I), n is an integer defining the variable number of ring carbons carrying R₁₀ and R₁₀ₐ and is selected from 1 to 4. In one embodiment, n is 1. In one embodiment, n is 2. In one embodiment, n is 3. In one embodiment, n is 4.

Exemplary compounds of formula (I) are those wherein at least one of R₄ or R₅ is selected from hydroxy, —OR₁₁, —OC(O)R₁₁, —OC(O)OR₁₁, —OSO₂R₁₁, —OC(O)NR₁₁R₁₂, —OP(O)(OH)₂, and —OP(O)(OH)OP(O)(OH)₂.

Other exemplary compounds of formula (I) are those wherein at least one of R₄ or R₅ is selected from hydroxy and —OC(O)R₁₁.

Other exemplary compounds of formula (I) are those wherein R₁₁ is an optionally substituted C₁-C₆ alkyl.

Other exemplary compounds of formula (I) are those wherein R₁₁ is an unsubstituted C₁-C₆ alkyl.

Other exemplary compounds of formula (I) are those wherein R₁₁ is methyl.

Other exemplary compounds of formula (I) are those wherein R₃ is selected from optionally substituted C₁-C₆ alkyl and optionally substituted C₂-C₆ alkenyl.

Other exemplary compounds of formula (I) are those wherein R₃ is an unsubstituted C₁-C₆ alkyl.

Other exemplary compounds of formula (I) are those wherein R₃ is methyl.

Other exemplary compounds of formula (I) are those wherein R₃ₐ is an electron pair.

Other exemplary compounds of formula (I) are those wherein R₆ is hydrogen.

Other exemplary compounds of formula (I) are those wherein R₇ is hydrogen.

Other exemplary compounds of formula (I) are those wherein R₉ is hydrogen.

Other exemplary compounds of formula (I) are those wherein R₁ is selected from hydrogen and optionally substituted C₁-C₆ alkyl.

Other exemplary compounds of formula (I) are those wherein R₁ is a 2-(S)-methyl-2-N,N-dimethylamino-ethyl or 2-(R)-methyl-2-N,N-dimethylamino-ethyl.

Other exemplary compounds of formula (I) are those wherein R₁₀ is hydrogen.

Other exemplary compounds of formula (I) are those wherein R₁₀, is hydrogen.

Other exemplary compounds of formula (I) are those wherein R₈ is hydrogen.

Other exemplary compounds of formula (I) are those wherein R₈ₐ is hydrogen.

Other exemplary compounds of formula (I) are those wherein n=1.

Other exemplary compounds of formula (I) are those wherein n=2.

Other exemplary compounds of formula (I) are those wherein n=3.

Other exemplary compounds of formula (I) are those wherein n=4.

Other exemplary compounds of formula (I) are those wherein R₂ is hydrogen.

Exemplary lead compounds of formula (I) can be found in Table 1.

TABLE 1

| Compound | Structural Formula |
|---|---|
| N-Methyl Aziridinyltryptamine Series | 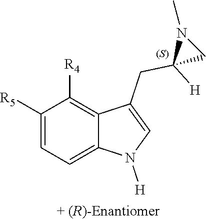 |
| | R₄ = OH: R₅ = H |
| | R₄= H; R₅ = OH |
| | R₄ = OAc; R₅ = H |
| | R₄ = H; R₅ = OAc |
| N-Methyl Azacyclobutyltryptamine Series | 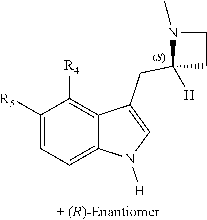 |
| | R₄ = OH: R₅ = H |
| | R₄= H; R₅ = OH |
| | R₄ = OAc; R₅ = H |
| | R₄ = H; R₅ = OAc |
| N-Methyl Azacyclopentyltryptamine Series | 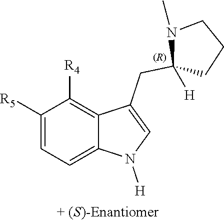 |
| | R₄ = OH: R₅ = H |
| | R₄= H; R₅ = OH |
| | R₄ = OAc; R₅ = H |
| | R₄ = H; R₅ = OAc |
| N-Methyl Azacyclohexyltryptamine Series | 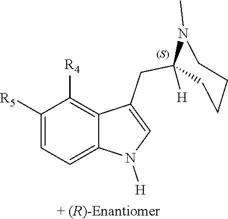 |
| | R₄ = OH: R₅ = H |
| | R₄= H; R₅ = OH |
| | R₄ = OAc; R₅ = H |
| | R₄ = H; R₅ = OAc |

Tryptamine analogs with endocyclic amines such as those represented in formula (I), can be prepared by synthesis of tryptamine analogs. The following reaction mechanism shows the synthesis of both 3-((1-methyl aziridin-2-yl)methyl)-1H-indol-4-ol as well as the acetate prodrug 3-((1-methyl aziridin-2-yl)methyl)-1H-indol-4-yl acetate. 4-Acetoxyindole can be alkylated at the 3-position to provide the intermediate aldehyde which is converted to the p-toluenesulfonimine by addition of the corresponding sulfonamide. The aziridine moiety is created by the action of in situ generated iodomethyl lithium (in this case where R₁ and R₂═H). (Concellón) The N-tosyl bond is cleaved through direct reduction to produce the secondary aziridine which can be alkylated with methyl iodide to produce the target compound (the 4-acetoxy derivative) which can be converted to the 4-hydroxy compound through saponification of the following reaction mechanism.

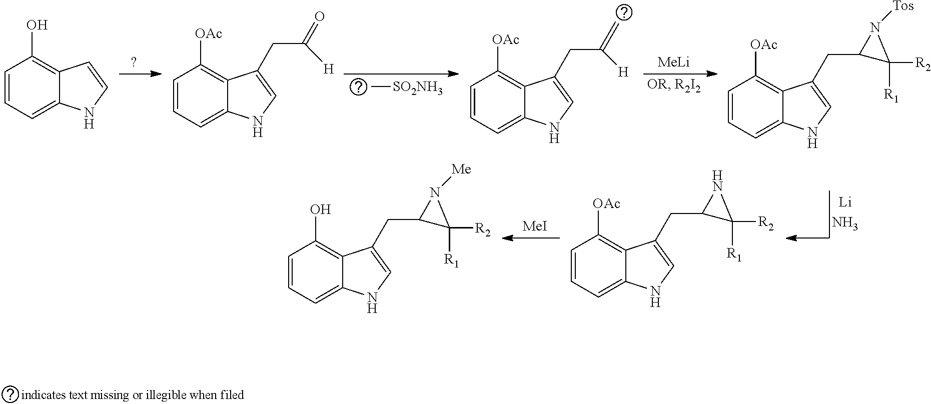

Formula (II) below depicts derivatives that provide for further optionality for the modulation of effect.

The disclosure further relates to a compound of formula (II):

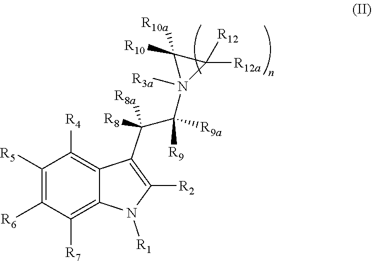

wherein:
   R₁ is selected from hydrogen, optionally substituted C₁-C₆ alkyl, optionally substituted C₂-C₆ alkenyl, optionally substituted aryl, —OR₁₁, —C(O)R₁₁, —C(O)OR₁₁, —SO₂R₁₁, and —C(O)NR₁₁R₁₃;
   R₂, R₄, R₅, R₆, R₇, R₈, R₈ₐ, R₉, R₉ₐ, R₁₀, R₁₀ₐ, R₁₂, and R₁₂ₐ are for each occurrence independently selected from hydrogen, optionally substituted C₁-C₆ alkyl, optionally substituted C₂-C₆ alkenyl, optionally substituted C₁-C₆-heteroalkyl, optionally substituted heteroaryl, optionally substituted aryl, halogen, hydroxy, CN, —OR₁₁, —OC(O)R₁₁, —OC(O)OR₁₁, OSO₂R₁₁, —OC(O)N R₁₁R₁₃, —OP(O)(OH)₂, and —OP(O)(OH)OP(O)(OH)₂;
   R₃ₐ is selected from an electron pair, hydrogen, optionally substituted C₁-C₆ alkyl, optionally substituted C₂-C₆ alkenyl, and optionally substituted aryl, wherein when R₃ₐ is not an electron pair the compound of formula (II) is a quaternary amine cation with a pharmaceutically acceptable anion, X⁻;
   R₁₁ and R₁₃ are for each occurrence independently selected from hydrogen, optionally substituted C₁-C₆ alkyl, optionally substituted C₂-C₆ alkenyl, and optionally substituted aryl; and
   n is an integer defining the variable number of ring carbons carrying R₁₂ and R₁₂ₐ and is selected from 1 to 4.

In formula (II), R₁ is selected from hydrogen, optionally substituted C₁-C₆ alkyl, optionally substituted C₂-C₆ alkenyl, optionally substituted aryl, —OR₁₁, —C(O)R₁₁, —C(O)OR₁₁, —SO₂R₁₁, and —C(O)NR₁₁R₁₃. R₁ may be hydrogen. R₁ may be an optionally substituted straight chain or branched C₁-C₆ alkyl, for example a straight chain C₁-C₆ alkyl, or an optionally substituted straight chain or branched C₂-C₆ alkenyl, for example allyl, 2-butenyl, etc. In some embodiments R₁ may be a straight chain or branched C₁-C₄ alkyl, for example a straight chain C₁-C₄ alkyl, or a C₂-C₄ alkenyl. R₁ may be an optionally substituted aryl. An aryl is a 6- to 14-membered aromatic ring, preferably a 6- to 10-membered aromatic ring and includes polycyclic ring systems in which two or more carbon atoms are common to adjoining rings where at least one ring is aromatic. Examples of aryl groups include, but are not limited to phenyl, naphthyl, anthracenyl, and phenantherenyl. An aryl group may be substituted with one or more C₁-C₄ alkyl or perfluoralkyl groups, C₁-C₄ hydroxyalkyl groups, hydroxyl groups, nitro groups or halo groups (e.g., F, Cl, I, or Br). An aryl group may be ortho-, meta-, and/or para-substituted, preferably para-substituted. R₁ may be —OR₁₁, —C(O)R₁₁, —C(O)OR₁₁, —SO₂R₁₁, or —C(O)N R₁₁R₁₃, wherein R₁₁ and R₁₃ are for each occurrence independently selected from hydrogen, optionally substituted C₁-C₆ alkyl, optionally substituted C₂-C₆ alkenyl, and optionally substituted aryl.

In formula (II), R₂, R₄, R₅, R₆, R₇, R₈, R₈ₐ, R₉, R₉ₐ, R₁₀, R₁₀ₐ, R₁₂, and R₁₂ₐ are for each occurrence independently selected from hydrogen, optionally substituted C₁-C₆ alkyl, optionally substituted C₂-C₆ alkenyl, optionally substituted C₁-C₆-heteroalkyl, optionally substituted heteroaryl, optionally substituted aryl, halogen, hydroxy, CN, —OR₁₁, —OC(O)R₁₁, —OC(O)OR₁₁, —OSO₂R₁₁, —OC(O)NR₁₁R₁₃, —OP(O)(OH)₂, and —OP(O)(OH)OP(O)(OH)₂. R₂, R₄, R₅, R₆, R₇, R₈, R₈ₐ, R₉, R₉ₐ, R₁₀, R₁₀ₐ, R₁₂, and R₁₂ₐ may each independently be hydrogen. R₂, R₄, R₅, R₆, R₇, R₈, R₈ₐ, R₉, R₉ₐ, R₁₀, R₁₀ₐ, R₁₂, and R₁₂ₐ may each independently be an optionally substituted straight chain or branched C₁-C₆ alkyl, for example a straight chain C₁-C₆ alkyl, or an optionally substituted straight chain or branched C₂-C₆ alkenyl, for example allyl, 2-butenyl, etc. In some embodiments R₂, R₄, R₅, R₆, R₇, R₈, R₈ₐ, R₉, R₉ₐ, R₁₀, R₁₀ₐ, R₁₂, and R₁₂ₐ may each independently be a straight chain or branched C₁-C₄ alkyl, for example a straight chain C₁-C₄ alkyl, or a C₂-C₄ alkenyl. R₂, R₄, R₅, R₆, R₇, R₈, R₈ₐ, R₉, R₉ₐ, R₁₀, R₁₀ₐ, R₁₂, and R₁₂ₐ may each independently be an optionally substituted C₁-C₆-heteroalkyl, including but not limited to alkoxy, alkylthio, and alkylamino. R₂, R₄, R₅, R₆, R₇, R₈, R₈ₐ, R₉, R₉ₐ, R₁₀, R₁₀ₐ, R₁₂, and R₁₂ₐ may each independently be an optionally substituted heteroaryl, including, but not limited to, furano, pyridinyl, pyrimidinyl, etc. R₂, R₄, R₅, R₆, R₇, R₈, R₈ₐ, R₉, R₉ₐ, R₁₀, R₁₀ₐ, R₁₂, and R₁₂ₐ may each independently be an optionally substituted aryl. An aryl is a 6- to 14-membered aromatic ring, preferably a 6- to 10-membered aromatic ring and includes polycyclic ring systems in which two or more carbon atoms are common to adjoining rings where at least one ring is aromatic. Examples of aryl groups include, but are not limited to phenyl, naphthyl, anthracenyl, and phenantherenyl. An aryl group may be substituted with one or more C₁-C₄ alkyl or perfluoralkyl groups, C₁-C₄ hydroxyalkyl groups, hydroxyl groups, nitro groups or halo groups (e.g., F, Cl, I, or Br). An aryl group may be ortho-, meta-, and/or para-substituted, preferably para-substituted. R₂, R₄, R₅, R₆, R₇, R₈, R₈ₐ, R₉, R₉ₐ, R₁₀, R₁₀ₐ, R₁₂, and R₁₂ₐ may each independently be a halogen. Exemplary halogens include fluorine (F), chlorine (Cl), bromine (Br), and iodine (I). R₂, R₄, R₅, R₆, R₇, R₈, R₈ₐ, R₉, R₉ₐ, R₁₀, R₁₀ₐ, R₁₂, and R₁₂ₐ may each independently by hydroxy. R₂, R₄, R₅, R₆, R₇, R₈, R₈ₐ, R₉, R₉ₐ, R₁₀, R₁₀ₐ, R₁₂, and R₁₂ₐ may each independently be CN. R₂, R₄, R₅, R₆, R₇, R₈, R₈ₐ, R₉, R₉ₐ, R₁₀, R₁₀ₐ, R₁₂, and R₁₂ₐ may each independently be —OR₁₁, —OC(O)R₁₁, —OC(O)OR₁₁, —OSO₂R₁₁, —OC(O)NR₁₁R₁₃, —OP(O)(OH)₂, or —OP(O)(OH)OP(O)(OH)₂, wherein R₁₁ and R₁₃ are for each occurrence independently selected from hydrogen, optionally substituted C₁-C₆ alkyl, optionally substituted C₂-C₆ alkenyl, and optionally substituted aryl.

In formula (II), R₃ₐ is selected from an electron pair, hydrogen, optionally substituted C₁-C₆ alkyl, optionally substituted C₂-C₆ alkenyl, and optionally substituted aryl, wherein when R₃ₐ is not an electron pair the compound of formula (II) is a quaternary amine cation with a pharmaceutically acceptable anion, X⁻. R₃ₐ may be an electron pair. R₃ₐ may be hydrogen. R₃ₐ may be an optionally substituted straight chain or branched C₁-C₆ alkyl, for example a straight chain C₁-C₆ alkyl, or an optionally substituted straight chain or branched C₂-C₆ alkenyl, for example allyl, 2-butenyl, etc. In some embodiments R₃ₐ may be a straight chain or branched C₁-C₄ alkyl, for example a straight chain C₁-C₄ alkyl, or a C₂-C₄ alkenyl. R₃ₐ may be an optionally substituted aryl. An aryl is a 6- to 14-membered aromatic ring, preferably a 6- to 10-membered aromatic ring and includes polycyclic ring systems in which two or more carbon atoms are common to adjoining rings where at least one ring is aromatic. Examples of aryl groups include, but are not limited to phenyl, naphthyl, anthracenyl, and phenantherenyl. An aryl group may be substituted with one or more C₁-C₄ alkyl or perfluoralkyl groups, C₁-C₄ hydroxyalkyl groups, hydroxyl groups, nitro groups or halo groups (e.g., F, Cl, I, or Br). An aryl group may be ortho-, meta-, and/or para-substituted, preferably para-substituted.

The anion of formula (II), X⁻, may be any pharmaceutically acceptable anion, for example, Cl⁻, I⁻, Br, ascorbate, hydrofumarate, fumarate, maleate, and the like.

In formula (II), R₁₁ and R₁₃ are for each occurrence independently selected from hydrogen, optionally substituted C₁-C₆ alkyl, optionally substituted C₂-C₆ alkenyl, and optionally substituted aryl. R₁₁ and R₁₃ may each independently be hydrogen. R₁₁ and R₁₃ may each independently be an optionally substituted straight chain or branched C₁-C₆ alkyl, for example a straight chain C₁-C₆ alkyl, or an optionally substituted straight chain or branched C₂-C₆ alkenyl, for example allyl, 2-butenyl, etc. In some embodiments R₁₁ and R₁₃ may each independently be a straight chain or branched C₁-C₄ alkyl, for example a straight chain C₁-C₄ alkyl, or a C₂-C₄ alkenyl. R₁₁ and R₁₃ may each independently be an optionally substituted aryl. An aryl is a 6- to 14-membered aromatic ring, preferably a 6- to 10-membered aromatic ring and includes polycyclic ring systems in which two or more carbon atoms are common to adjoining rings where at least one ring is aromatic. Examples of aryl groups include, but are not limited to phenyl, naphthyl, anthracenyl, and phenantherenyl. An aryl group may be substituted with one or more C₁-C₄ alkyl or perfluoralkyl groups, C₁-C₄ hydroxyalkyl groups, hydroxyl groups, nitro groups or halo groups (e.g., F, Cl, I, or Br). An aryl group may be ortho-, meta-, and/or para-substituted, preferably para-substituted.

In formula (II), n is an integer defining the variable number of ring carbons carrying R₁₂ and R₁₂ₐ and is selected from 1 to 4. In one embodiment, n is 1. In one embodiment, n is 2. In one embodiment, n is 3. In one embodiment, n is 4.

Exemplary compounds of formula (II) are those with the proviso that when n=2, R₃ₐ is not an electron pair and/or at least one of R₈, R₈ₐ, R₉, or R₉ₐ is not hydrogen.

Other exemplary compounds of formula (II) are those wherein at least one of R₄ or R₅ is selected from hydroxy, —OR₁₁, —OC(O)R₁₁, —OC(O)OR₁₁, —OSO₂R₁₁, —OC(O)NR₁₁R₁₂, —OP(O)(OH)₂, and —OP(O)(OH)OP(O)(OH)₂.

Other exemplary compounds of formula (II) are those wherein at least one of R₄ or R₅ is selected from hydroxy and —OC(O)R₁₁.

Other exemplary compounds of formula (II) are those wherein R₁₁ is an optionally substituted C₁-C₆ alkyl.

Other exemplary compounds of formula (II) are those wherein R₁₁ is an unsubstituted C₁-C₆ alkyl.

Other exemplary compounds of formula (II) are those wherein R₁₁ is methyl.

Other exemplary compounds of formula (II) are those wherein R₃ₐ is an electron pair.

Other exemplary compounds of formula (II) are those wherein R₆ is hydrogen.

Other exemplary compounds of formula (II) are those wherein R₇ is hydrogen.

Other exemplary compounds of formula (II) are those wherein R₉ is hydrogen.

Other exemplary compounds of formula (II) are those wherein R₉ₐ is hydrogen.

Other exemplary compounds of formula (II) are those wherein R₈ is hydrogen.

Other exemplary compounds of formula (II) are those wherein R₈ₐ is hydrogen.

Other exemplary compounds of formula (II) are those wherein R₁ is selected from hydrogen and optionally substituted C₁-C₆ alkyl.

Other exemplary compounds of formula (II) are those wherein R₁ is selected from 2-(S)-methyl-2-N,N-dimethylamino-ethyl or 2-(R)-methyl-2-N,N-dimethylamino-ethyl.

Other exemplary compounds of formula (II) are those wherein R₁₀ is hydrogen.

Other exemplary compounds of formula (II) are those wherein R₁₀ₐ is hydrogen.

Other exemplary compounds of formula (II) are those wherein R₁₂ is hydrogen.

Other exemplary compounds of formula (II) are those wherein R₁₂ₐ is hydrogen.

Other exemplary compounds of formula (II) are those wherein n=1.

Other exemplary compounds of formula (II) are those wherein n=2.

Other exemplary compounds of formula (II) are those wherein n=2, with the proviso that R₃ₐ is not an electron pair and/or at least one of R₈, R₈ₐ, R₉, or R₉ₐ is not hydrogen.

Other exemplary compounds of formula (II) are those wherein n=3.

Other exemplary compounds of formula (II) are those wherein n=4.

An exemplary compound of formula (II):

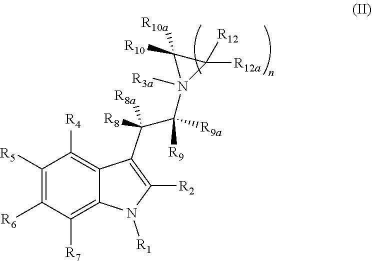

wherein:
   R₁ is selected from hydrogen, optionally substituted C₁-C₆ alkyl, optionally substituted C₂-C₆ alkenyl, optionally substituted aryl, —OR₁₁, —C(O)R₁₁, —C(O)OR₁₁, —SO₂R₁₁, and —C(O)NR₁₁R₁₃;
   R₂, R₄, R₅, R₆, R₇, R₈, R₈ₐ, R₉, R₉ₐ, R₁₀, R₁₀ₐ, R₁₂, and R₁₂ₐ are for each occurrence independently selected from hydrogen, optionally substituted C₁-C₆ alkyl, optionally substituted C₂-C₆ alkenyl, optionally substituted C₁-C₆-heteroalkyl, optionally substituted heteroaryl, optionally substituted aryl, halogen, hydroxy, CN, —OR₁₁, —OC(O)R₁₁, —OC(O)OR₁₁, OSO₂R₁₁, —OC(O)N R₁₁R₁₃, —OP(O)(OH)₂, and —OP(O)(OH)OP(O)(OH)₂;
   R₃ₐ is selected from an electron pair, hydrogen, optionally substituted C₁-C₆ alkyl, optionally substituted C₂-C₆ alkenyl, and optionally substituted aryl, wherein when R₃ₐ is not an electron pair the compound of formula (II) is a quaternary amine cation with a pharmaceutically acceptable anion, X⁻;
   R₁₁ and R₁₃ are for each occurrence independently selected from hydrogen, optionally substituted C₁-C₆ alkyl, optionally substituted C₂-C₆ alkenyl, and optionally substituted aryl; and
   n is an integer defining the variable number of ring carbons carrying R₁₂ and R₁₂ₐ and is selected from 1 to 4, with the proviso that when n=2, R₃ₐ is not an electron pair and/or at least one of R₈, R₈ₐ, R₉, or R₉ₐ is not hydrogen.

Exemplary lead compounds of formula (II) can be found in Table 2.

TABLE 2

| Compound | Structural Formula |
|---|---|
| Aziridinyltryptamine Series | 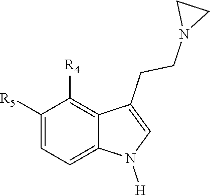 |
| | R₄ = OH: R₅ = H |
| | R₄= H; R₅ = OH |
| | R₄ = OAc; R₅ = H |
| | R₄ = H; R₅ = OAc |
| Azacyclopentyltryptamine Series | 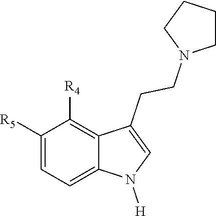 |
| | R₄ = OH: R₅ = H |
| | R₄= H; R₅ = OH |
| | R₄ = OAc; R₅ = H |
| | R₄ = H; R₅ = OAc |
| Azacyclohexyltryptamine Series | 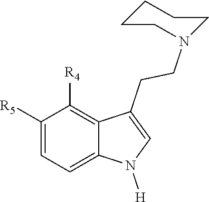 |
| | R₄ = OH: R₅ = H |
| | R₄= H; R₅ = OH |
| | R₄ = OAc; R₅ = H |
| | R₄ = H; R₅ = OAc |

Tryptamine analogs with exocyclic amines, such as those represented in formula (II), are easily prepared by the venerable Speeter-Anthony synthesis of tryptamine analogs. (Speeter) The following reaction mechanism shows the synthesis of both 3-(2-(azetidin-1-yl)ethyl)-1H-indol-4-yl acetate as well as 3-(2-(azetidin-1-yl)ethyl)-1H-indol-4-ol. Widely available 4-hydroxyindole is acetylated to provide 4-acetoxyindole. Alternatively, 4-acetoxy-indole, since it is also widely available, may be purchased as the starting material. 4-Acetoxyindole is acylated at the 3-position with oxalyl chloride to provide the intermediate acyl chloride which is converted to the terminal amide by addition of the cyclic amine, in this case, azacyclobutane. The carbonyls are readily reduced by the action of lithium aluminum hydride to provide the acetoxy tryptamine derivative. This compound can be saponified to provide the 4-hydroxytryptamine derivative as shown in the following reaction mechanism.

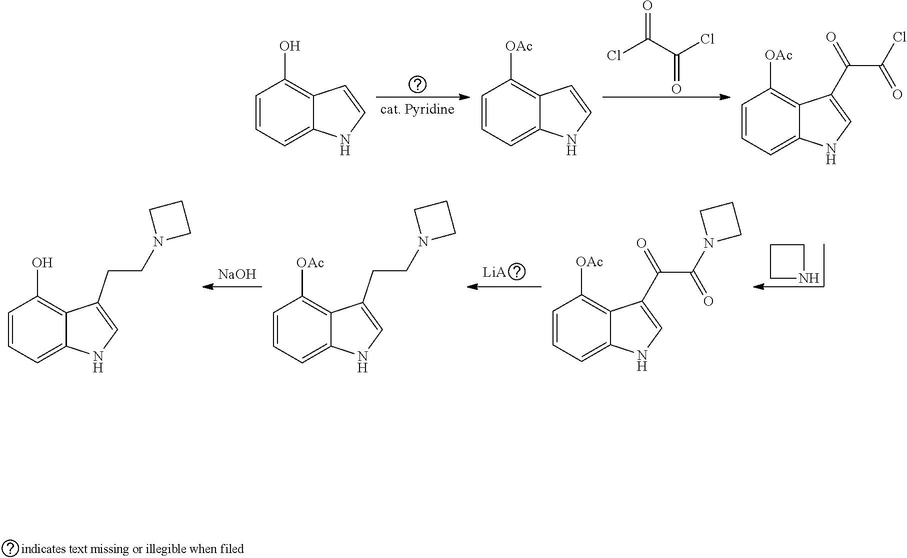

Other compounds of formula (II) can be formed in a similar manner.

It is reasoned that for MDMA, the binding affinity to and fidelity for critical receptors may be modulated by conformational and/or rotational restriction about the moieties that bind the receptor sites. In addition, changes in C—N—C atomic bond lengths and nitrogen hybridization and barrier to pyramidal inversion can also be levers to adjust pharmacological properties. (Padwa; Katritzky) To that end, derivatives of MDMA have been designed and the following Markush structure depicting the scope of such derivatives is shown in formula (III) below.

A key feature of this molecular genus is the appearance of small ring heterocycles which change bond lengths and angles in the α-methyl-N-methylethylamine moiety of MDMA. Additional modulation of activity can be brought about through substitutions on the aryl ring and other portions of the molecule with different elements.

The disclosure further relates to a compound of formula (III):

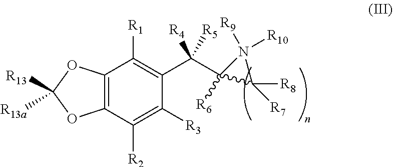

wherein:
   R₁, R₂, and R₃ are independently selected from hydrogen, optionally substituted C₁-C₆ alkyl, optionally substituted C₂-C₆ alkenyl, optionally substituted aryl, halogen, —OR₁₁, —C(O)R₁₁, —C(O)OR₁₁, —OC(O)R₁₁, C(O)NR₁₁R₁₂, CN, C(NR₁₂)R₁₁, C(NOR₁₂)OR₁₁, —SO₂R₁₁, and —C(O)N R₁₁R₁₂;
   R₄, R₅, R₆, R₇, and R₈ are for each occurrence independently selected from hydrogen, optionally substituted C₁-C₆ alkyl, optionally substituted C₂-C₆ alkenyl, optionally substituted C₁-C₆ heteroalkyl, optionally substituted heteroaryl, optionally substituted aryl, halogen, hydroxy, —OR₁₁, —OC(O)R₁₁, —OC(O)OR₁₁, —OSO₂R₁₁, and —OC(O)NR₁₁R₁₂;
   R₉ is selected from hydrogen, optionally substituted C₁-C₆ alkyl, optionally substituted C₂-C₆ alkenyl, optionally substituted aryl, and —SO₂R₁₁;
   R₁₀ is selected from an electron pair, hydrogen, optionally substituted C₁-C₆ alkyl, optionally substituted C₂-C₆ alkenyl, and optionally substituted aryl, wherein when R₁₀ is not an electron pair the compound of formula (III) is a quaternary amine cation with a pharmaceutically acceptable anion, X⁻;
   R₁₁ and R₁₂ are for each occurrence independently selected from hydrogen, optionally substituted C₁-C₆ alkyl, optionally substituted C₂-C₆ alkenyl, and optionally substituted aryl;
   R₁₃ and R₁₃ₐ are for each occurrence independently selected from hydrogen, optionally substituted C₁-C₆ alkyl, optionally substituted C₂-C₆ alkenyl, and optionally substituted aryl, or R₁₃ and R₁₃ₐ are taken together with the carbon to which they are bound to form a 3- to 6-membered cycloalkyl; and
   n is an integer defining the variable number of ring carbons carrying R₇ and R₈ and is selected from 1 to 4.

In formula (III), R₁, R₂, and R₃ are independently selected from hydrogen, optionally substituted C₁-C₆ alkyl, optionally substituted C₂-C₆ alkenyl, optionally substituted aryl, halogen, —OR₁₁, —C(O)R₁₁, —C(O)OR₁₁, —OC(O)R₁₁, C(O)NR₁₁R₁₂, CN, C(NR₁₂)R₁₁, C(NOR₁₂)OR₁₁, —SO₂R₁₁, and —C(O)N R₁₁R₁₂. R₁, R₂, and R₃ may each independently be hydrogen. R₁, R₂, and R₃ may each independently be an optionally substituted straight chain or branched C₁-C₆ alkyl, for example a straight chain C₁-C₆ alkyl, or an optionally substituted straight chain or branched C₂-C₆ alkenyl, for example allyl, 2-butenyl, etc. In some embodiments R₁, R₂, and R₃ may each independently be a straight chain or branched C₁-C₄ alkyl, for example a straight chain C₁-C₄ alkyl, or a C₂-C₄ alkenyl. R₁, R₂, and R₃ may each independently be an optionally substituted aryl. An aryl is a 6- to 14-membered aromatic ring, preferably a 6- to 10-membered aromatic ring and includes polycyclic ring systems in which two or more carbon atoms are common to adjoining rings where at least one ring is aromatic. Examples of aryl groups include, but are not limited to phenyl, naphthyl, anthracenyl, and phenantherenyl. An aryl group may be substituted with one or more C₁-C₄ alkyl or perfluoralkyl groups, C₁-C₄ hydroxyalkyl groups, hydroxyl groups, nitro groups or halo groups (e.g., F, Cl, I, or Br). An aryl group may be ortho-, meta-, and/or para-substituted, preferably para-substituted. R₁, R₂, and R₃ may each independently be a halogen. Exemplary halogens include fluorine (F), chlorine (Cl), bromine (Br), and iodine (I). R₁, R₂, and R₃ may each independently be —OR₁₁, —C(O)R₁₁, —C(O)OR₁₁, —OC(O)R₁₁, C(O)NR₁₁R₁₂, CN, C(NR₁₂)R₁₁, C(NOR₁₂)OR₁₁, —SO₂R₁₁, and —C(O)N R₁₁R₁₂, wherein R₁₁ and R₁₂ are for each occurrence independently selected from hydrogen, optionally substituted C₁-C₆ alkyl, optionally substituted C₂-C₆ alkenyl, and optionally substituted aryl.

In formula (III), R₄, R₅, R₆, R₇, and R₈ are for each occurrence independently selected from hydrogen, optionally substituted C₁-C₆ alkyl, optionally substituted C₂-C₆ alkenyl, optionally substituted C₁-C₆ heteroalkyl, optionally substituted heteroaryl, optionally substituted aryl, halogen, hydroxy, —OR₁₁, —OC(O)R₁₁, —OC(O)OR₁₁, —OSO₂R₁₁, and —OC(O)N R₁₁R₁₂. R₄, R₅, R₆, R₇, and R₈ may each independently be hydrogen. R₄, R₅, R₆, R₇, and R₈ may each independently be an optionally substituted straight chain or branched C₁-C₆ alkyl, for example a straight chain C₁-C₆ alkyl, or an optionally substituted straight chain or branched C₂-C₆ alkenyl, for example allyl, 2-butenyl, etc. In some embodiments R₄, R₅, R₆, R₇, and R₈ may each independently be a straight chain or branched C₁-C₄ alkyl, for example a straight chain C₁-C₄ alkyl, or a C₂-C₄ alkenyl. R₄, R₅, R₆, R₇, and R₈ may each independently be an optionally substituted C₁-C₆-heteroalkyl, including but not limited to alkoxy, alkylthio, and alkylamino. R₄, R₅, R₆, R₇, and R₈ may each independently be an optionally substituted heteroaryl, including, but not limited to, furano, pyridinyl, pyrimidinyl, etc. R₄, R₅, R₆, R₇, and R₈ may each independently be an optionally substituted aryl. An aryl is a 6- to 14-membered aromatic ring, preferably a 6- to 10-membered aromatic ring and includes polycyclic ring systems in which two or more carbon atoms are common to adjoining rings where at least one ring is aromatic. Examples of aryl groups include, but are not limited to phenyl, naphthyl, anthracenyl, and phenantherenyl. An aryl group may be substituted with one or more C₁-C₄ alkyl or perfluoralkyl groups, C₁-C₄ hydroxyalkyl groups, hydroxyl groups, nitro groups or halo groups (e.g., F, Cl, I, or Br). An aryl group may be ortho-, meta-, and/or para-substituted, preferably para-substituted. R₄, R₅, R₆, R₇, and R₈ may each independently be a halogen. Exemplary halogens include fluorine (F), chlorine (Cl), bromine (Br), and iodine (I). R₄, R₅, R₆, R₇, and R₈ may each independently be hydroxy. R₄, R₅, R₆, R₇, and R₈ may each independently be —OR₁₁, —OC(O)R₁₁, —OC(O)OR₁₁, —OSO₂R₁₁, or —OC(O)N R₁₁R₁₂, wherein R₁₁ and R₁₂ are for each occurrence independently selected from hydrogen, optionally substituted C₁-C₆ alkyl, optionally substituted C₂-C₆ alkenyl, and optionally substituted aryl.

In formula (III), R₉ is selected from hydrogen, optionally substituted C₁-C₆ alkyl, optionally substituted C₂-C₆ alkenyl, optionally substituted aryl, and —SO₂Rn. R₉ may be hydrogen. R₉ may be an optionally substituted straight chain or branched C₁-C₆ alkyl, for example a straight chain C₁-C₆ alkyl, or an optionally substituted straight chain or branched C₂-C₆ alkenyl, for example allyl, 2-butenyl, etc. In some embodiments R₉ may be a straight chain or branched C₁-C₄ alkyl, for example a straight chain C₁-C₄ alkyl, or a C₂-C₄ alkenyl. R₉ may be an optionally substituted aryl. An aryl is a 6- to 14-membered aromatic ring, preferably a 6- to 10-membered aromatic ring and includes polycyclic ring systems in which two or more carbon atoms are common to adjoining rings where at least one ring is aromatic. Examples of aryl groups include, but are not limited to phenyl, naphthyl, anthracenyl, and phenantherenyl. An aryl group may be substituted with one or more C₁-C₄ alkyl or perfluoralkyl groups, C₁-C₄ hydroxyalkyl groups, hydroxyl groups, nitro groups or halo groups (e.g., F, Cl, I, or Br). An aryl group may be ortho-, meta-, and/or para-substituted, preferably para-substituted. R₉ may be —SO₂R₁₁, wherein R₁ may be selected from hydrogen, optionally substituted C₁-C₆ alkyl, optionally substituted C₂-C₆ alkenyl, and optionally substituted aryl.

In formula (III), R₁₀ is selected from an electron pair, hydrogen, optionally substituted C₁-C₆ alkyl, optionally substituted C₂-C₆ alkenyl, and optionally substituted aryl, wherein when R₁₀ is not an electron pair the compound of formula (III) is a quaternary amine cation with a pharmaceutically acceptable anion, X⁻. R₁₀ may be an electron pair. R₁₀ may be hydrogen. R₁₀ may be an optionally substituted straight chain or branched C₁-C₆ alkyl, for example a straight chain C₁-C₆ alkyl, or an optionally substituted straight chain or branched C₂-C₆ alkenyl, for example allyl, 2-butenyl, etc. In some embodiments R₁₀ may be a straight chain or branched C₁-C₄ alkyl, for example a straight chain C₁-C₄ alkyl, or a C₂-C₄ alkenyl. R₁₀ may be an optionally substituted aryl. An aryl is a 6- to 14-membered aromatic ring, preferably a 6- to 10-membered aromatic ring and includes polycyclic ring systems in which two or more carbon atoms are common to adjoining rings where at least one ring is aromatic. Examples of aryl groups include, but are not limited to phenyl, naphthyl, anthracenyl, and phenantherenyl. An aryl group may be substituted with one or more C₁-C₄ alkyl or perfluoralkyl groups, C₁-C₄ hydroxyalkyl groups, hydroxyl groups, nitro groups or halo groups (e.g., F, Cl, I, or Br). An aryl group may be ortho-, meta-, and/or para-substituted, preferably para-substituted.

The anion of formula (III), X⁻, may be any pharmaceutically acceptable anion, for example, Cl⁻, I⁻, Br⁻, ascorbate, hydrofumarate, fumarate, maleate, and the like.

In formula (III), R₁₁ and R₁₂ are for each occurrence independently selected from hydrogen, optionally substituted C₁-C₆ alkyl, optionally substituted C₂-C₆ alkenyl, and optionally substituted aryl. R₁₁ and R₁₂ may each independently be hydrogen. R₁₁ and R₁₂ may each independently be an optionally substituted straight chain or branched C₁-C₆ alkyl, for example a straight chain C₁-C₆ alkyl, or an optionally substituted straight chain or branched C₂-C₆ alkenyl, for example allyl, 2-butenyl, etc. In some embodiments R₁₁ and R₁₂ may each independently be a straight chain or branched C₁-C₄ alkyl, for example a straight chain C₁-C₄ alkyl, or a C₂-C₄ alkenyl. R₁₁ and R₁₂ may each independently be an optionally substituted aryl. An aryl is a 6- to 14-membered aromatic ring, preferably a 6- to 10-membered aromatic ring and includes polycyclic ring systems in which two or more carbon atoms are common to adjoining rings where at least one ring is aromatic. Examples of aryl groups include, but are not limited to phenyl, naphthyl, anthracenyl, and phenantherenyl. An aryl group may be substituted with one or more C₁-C₄ alkyl or perfluoralkyl groups, C₁-C₄ hydroxyalkyl groups, hydroxyl groups, nitro groups or halo groups (e.g., F, Cl, I, or Br). An aryl group may be ortho-, meta-, and/or para-substituted, preferably para-substituted.

In formula (III), R₁₃ and R₁₃ₐ are for each occurrence independently selected from hydrogen, optionally substituted C₁-C₆ alkyl, optionally substituted C₂-C₆ alkenyl, and optionally substituted aryl, or R₁₃ and R₁₃ₐ are taken together with the carbon to which they are bound to form a 3- to 6-membered cycloalkyl. R₁₃ and R₁₃ₐ may each independently be hydrogen. R₁₃ and R₁₃ₐ may each independently be an optionally substituted straight chain or branched C₁-C₆ alkyl, for example a straight chain C₁-C₆ alkyl, or an optionally substituted straight chain or branched C₂-C₆ alkenyl, for example allyl, 2-butenyl, etc. In some embodiments R₁₃ and R₁₃ₐ may each independently be a straight chain or branched C₁-C₄ alkyl, for example a straight chain C₁-C₄ alkyl, or a C₂-C₄ alkenyl. R₁₃ and R₁₃ₐ may each independently be an optionally substituted aryl. An aryl is a 6- to 14-membered aromatic ring, preferably a 6- to 10-membered aromatic ring and includes polycyclic ring systems in which two or more carbon atoms are common to adjoining rings where at least one ring is aromatic. Examples of aryl groups include, but are not limited to phenyl, naphthyl, anthracenyl, and phenantherenyl. An aryl group may be substituted with one or more C₁-C₄ alkyl or perfluoralkyl groups, C₁-C₄ hydroxyalkyl groups, hydroxyl groups, nitro groups or halo groups (e.g., F, Cl, I, or Br). An aryl group may be ortho-, meta-, and/or para-substituted, preferably para-substituted. R₁₃ and R₁₃ₐ may be taken together with the carbon to which they are bound to form a 3- to 6-membered cycloalkyl. In some embodiments, R₁₃ and R₁₃ₐ may be taken together with the carbon to which they are bound to form cyclopropyl, cyclobutyl, cyclopentyl, or cyclohexyl.

In formula (III), n is an integer defining the variable number of ring carbons carrying R₇ and R₈ and is selected from 1 to 4. In one embodiment, n is 1. In one embodiment, n is 2. In one embodiment, n is 3. In one embodiment, n is 4.

Exemplary compounds of formula (III) are those wherein R₁ is hydrogen.

Other exemplary compounds of formula (III) are those wherein R₂ is hydrogen.

Other exemplary compounds of formula (III) are those wherein R₃ is hydrogen.

Other exemplary compounds of formula (III) are those wherein R₄ is hydrogen.

Other exemplary compounds of formula (III) are those wherein Rₛ is hydrogen.

Other exemplary compounds of formula (III) are those wherein R₆ is hydrogen.

Other exemplary compounds of formula (III) are those wherein R₉ is an optionally substituted C₁-C₆ alkyl.

Other exemplary compounds of formula (III) are those wherein R₉ is an unsubstituted C₁-C₆ alkyl.

Other exemplary compounds of formula (III) are those wherein R₉ is methyl.

Other exemplary compounds of formula (III) are those wherein R₁₀ is an electron pair.

Other exemplary compounds of formula (III) are those wherein R₇ is hydrogen.

Other exemplary compounds of formula (III) are those wherein R₈ is hydrogen.

Other exemplary compounds of formula (III) are those wherein R₁₃ is hydrogen.

Other exemplary compounds of formula (III) are those wherein R₁₃ₐ is hydrogen.

Other exemplary compounds of formula (III) are those wherein R₁₃ and R₁₃ₐ are taken together with the carbon to which they are bound to form a cyclopropyl group.

Other exemplary compounds of formula (III) are those wherein R₁₃ and R₁₃ₐ are taken together with the carbon to which they are bound to form a cyclobutyl group.

Other exemplary compounds of formula (III) are those wherein R₁₃ and R₁₃ₐ are taken together with the carbon to which they are bound to form a cyclopentyl group.

Other exemplary compounds of formula (III) are those wherein R₁₃ and R₁₃ₐ are taken together with the carbon to which they are bound to form a cyclohexyl group.

Other exemplary compounds of formula (III) are those wherein n=1.

Other exemplary compounds of formula (III) are those wherein n=2.

Other exemplary compounds of formula (III) are those wherein n=3.

Other exemplary compounds of formula (III) are those wherein n=4.

Exemplary lead compounds of formula (III) can be found in Table 3.

TABLE 3

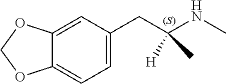

(S)-MDMA
(S)-MDMA Derivatives +
(R)-Enantiomer

| Compound | Structural Formula |
|---|---|
| Azacyclopropane Series | 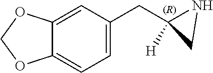 |
| Azcyclobutane Series | 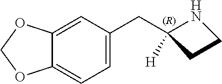 |
| Azacyclopentane Series | 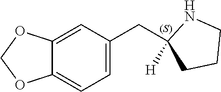 |
| Azacyclohexane Series | 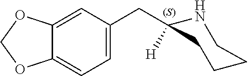 |

Commercially available piperonal (shown in the following reaction mechanism) can be converted to the vinyl azide in 72% yield in a single pot with first, reaction with ethynyl magnesium bromide to produce the in situ carbinol. A silver-(I) catalyzed hydroazidation yields the α,β-unsaturated-α-vinyl azide. The a-keto-aziridine is formed by treatment with potassium phosphate (K₃PO₄) in DMF at 95° C. in 86% yield. (Liu). Reduction of the aryl ketone to produce the target compound can be accomplished through a Wolff-Kishner reduction shown below or by any number of transformations known to those skilled in the art.

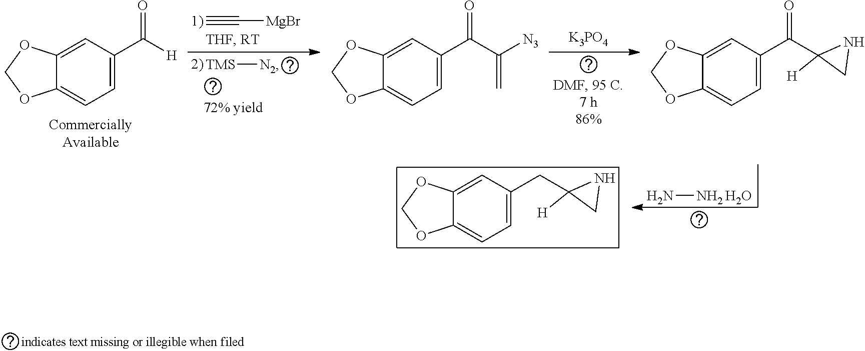

The disclosure further relates to a compound of formula (IV):

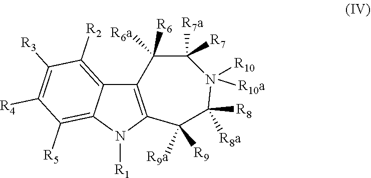

wherein:
   R₁ is selected from hydrogen, optionally substituted C₁-C₆ alkyl, optionally substituted C₂-C₆ alkenyl, optionally substituted aryl, —OR₁₁, —C(O)R₁₁, —C(O)OR₁₁, —SO₂R₁₁, and —C(O)NR₁₁R₁₁;
   R₂, R₃, R₄, R₅, R₆, R₆ₐ, R₇, R₇ₐ, R₈, R₈ₐ, R₉, and R₉ₐ are for each occurrence independently selected from hydrogen, optionally substituted C₁-C₆ alkyl, optionally substituted C₂-C₆ alkenyl, optionally substituted C₁-C₆-heteroalkyl, optionally substituted heteroaryl, optionally substituted aryl, halogen, hydroxy, —OR₁₁, —OC(O)R₁₁, —OC(O)OR₁₁, —OSO₂R₁₁, and —OC(O)N R₁₁R₁₂;
   R₁₀ is selected from an electron pair, hydrogen, optionally substituted C₁-C₆ alkyl, optionally substituted C₂-C₆ alkenyl, and optionally substituted aryl, wherein when R₁₀ is not an electron pair the compound of formula (IV) is a quaternary amine cation with a pharmaceutically acceptable anion, X⁻;
   R₁₀ₐ is taken together with one of R₈, R₈ₐ, R₉, or R₉ₐ to form optionally substituted 3- to 6-membered heterocyclic ring; and
   R₁₁ and R₁₂ are for each occurrence independently selected from hydrogen, optionally substituted C₁-C₆ alkyl, optionally substituted C₂-C₆ alkenyl, and optionally substituted aryl.

In formula (IV), R₁ is selected from hydrogen, optionally substituted C₁-C₆ alkyl, optionally substituted C₂-C₆ alkenyl, optionally substituted aryl, —OR₁₁, —C(O)R₁₁, —C(O)OR₁₁, —SO₂R₁₁, and —C(O)N R₁₁R₁₂. R₁ may be hydrogen. R₁ may be an optionally substituted straight chain or branched C₁-C₆ alkyl, for example a straight chain C₁-C₆ alkyl, or an optionally substituted straight chain or branched C₂-C₆ alkenyl, for example allyl, 2-butenyl, etc. In some embodiments R₁ may be a straight chain or branched C₁-C₄ alkyl, for example a straight chain C₁-C₄ alkyl, or a C₂-C₄ alkenyl. R₁ may be an optionally substituted aryl. An aryl is a 6- to 14-membered aromatic ring, preferably a 6- to 10-membered aromatic ring and includes polycyclic ring systems in which two or more carbon atoms are common to adjoining rings where at least one ring is aromatic. Examples of aryl groups include, but are not limited to phenyl, naphthyl, anthracenyl, and phenantherenyl. An aryl group may be substituted with one or more C₁-C₄ alkyl or perfluoralkyl groups, C₁-C₄ hydroxyalkyl groups, hydroxyl groups, nitro groups or halo groups (e.g., F, Cl, I, or Br). An aryl group may be ortho-, meta-, and/or para-substituted, preferably para-substituted. R₁ may be —OR₁₁, —C(O)R₁₁, —C(O)OR₁₁, —SO₂R₁₁, or —C(O)N R₁₁R₁₂, wherein R₁₁ and R₁₂ are for each occurrence independently selected from hydrogen, optionally substituted C₁-C₆ alkyl, optionally substituted C₂-C₆ alkenyl, and optionally substituted aryl.

In formula (IV), R₂, R₃, R₄, R₅, R₆, R₆ₐ, R₇, R₇ₐ, R₈, R₈ₐ, R₉, and R₉ₐ are for each occurrence independently selected from hydrogen, optionally substituted C₁-C₆ alkyl, optionally substituted C₂-C₆ alkenyl, optionally substituted C₁-C₆-heteroalkyl, optionally substituted heteroaryl, optionally substituted aryl, halogen, hydroxy, —OR₁₁, —OC(O)R₁₁, —OC(O)OR₁₁, —OSO₂R₁₁, and —OC(O)N R₁₁R₁₂. R₂, R₃, R₄, R₅, R₆, R₆ₐ, R₇, R₇ₐ, R₈, R₈ₐ, R₉, and R₉ₐ may each independently be hydrogen. R₂, R₃, R₄, R₅, R₆, R₆ₐ, R₇, R₇ₐ, R₈, R₈ₐ, R₉, and R₉ₐ may each independently be an optionally substituted straight chain or branched C₁-C₆ alkyl, for example a straight chain C₁-C₆ alkyl, or an optionally substituted straight chain or branched C₂-C₆ alkenyl, for example allyl, 2-butenyl, etc. In some embodiments R₂, R₃, R₄, R₅, R₆, R₆ₐ, R₇, R₇ₐ, R₈, R₈ₐ, R₉, and R₉ₐ may each independently be a straight chain or branched C₁-C₄ alkyl, for example a straight chain C₁-C₄ alkyl, or a C₂-C₄ alkenyl. R₂, R₃, R₄, R₅, R₆, R₆ₐ, R₇, R₇ₐ, R₈, R₈ₐ, R₉, and R₉ₐ may each independently be an optionally substituted C₁-C₆-heteroalkyl, including but not limited to alkoxy, alkylthio, and alkylamino. R₂, R₃, R₄, R₅, R₆, R₆ₐ, R₇, R₇ₐ, R₈, R₈ₐ, R₉, and R₉ₐ may each independently be an optionally substituted heteroaryl, including, but not limited to, furano, pyridinyl, pyrimidinyl, etc. R₂, R₃, R₄, R₅, R₆, R₆ₐ, R₇, R₇ₐ, R₈, R₈ₐ, R₉, and R₉ₐ may each independently be an optionally substituted aryl. An aryl is a 6- to 14-membered aromatic ring, preferably a 6- to 10-membered aromatic ring and includes polycyclic ring systems in which two or more carbon atoms are common to adjoining rings where at least one ring is aromatic. Examples of aryl groups include, but are not limited to phenyl, naphthyl, anthracenyl, and phenantherenyl. An aryl group may be substituted with one or more C₁-C₄ alkyl or perfluoralkyl groups, C₁-C₄ hydroxyalkyl groups, hydroxyl groups, nitro groups or halo groups (e.g., F, Cl, I, or Br). An aryl group may be ortho-, meta-, and/or para-substituted, preferably para-substituted. R₂, R₃, R₄, R₅, R₆, R₆ₐ, R₇, R₇ₐ, R₈, R₈ₐ, R₉, and R₉ₐ may each independently be a halogen. Exemplary halogens include fluorine (F), chlorine (Cl), bromine (Br), and iodine (I). R₂, R₃, R₄, R₅, R₆, R₆ₐ, R₇, R₇ₐ, R₈, R₈ₐ, R₉, and R₉ₐ may each independently be hydroxy. R₂, R₃, R₄, R₅, R₆, R₆ₐ, R₇, R₇ₐ, R₈, R₈ₐ, R₉, and R₉ₐ may each independently be —OR₁₁, —OC(O)R₁₁, —OC(O)OR₁₁, —OSO₂R₁₁, or —OC(O)N R₁₁R₁₂, wherein R₁₁ and R₁₂ are for each occurrence independently selected from hydrogen, optionally substituted C₁-C₆ alkyl, optionally substituted C₂-C₆ alkenyl, and optionally substituted aryl.

In formula (IV), R₁₀ is selected from an electron pair, hydrogen, optionally substituted C₁-C₆ alkyl, optionally substituted C₂-C₆ alkenyl, and optionally substituted aryl, wherein when R₁₀ is not an electron pair the compound of formula (IV) is a quaternary amine cation with a pharmaceutically acceptable anion, X⁻. R₁₀ may be an electron pair. R₁₀ may be hydrogen. R₁₀ may be an optionally substituted straight chain or branched C₁-C₆ alkyl, for example a straight chain C₁-C₆ alkyl, or an optionally substituted straight chain or branched C₂-C₆ alkenyl, for example allyl, 2-butenyl, etc. In some embodiments R₁₀ may be a straight chain or branched C₁-C₄ alkyl, for example a straight chain C₁-C₄ alkyl, or a C₂-C₄ alkenyl. R₁₀ may be an optionally substituted aryl. An aryl is a 6- to 14-membered aromatic ring, preferably a 6- to 10-membered aromatic ring and includes polycyclic ring systems in which two or more carbon atoms are common to adjoining rings where at least one ring is aromatic. Examples of aryl groups include, but are not limited to phenyl, naphthyl, anthracenyl, and phenantherenyl. An aryl group may be substituted with one or more C₁-C₄ alkyl or perfluoralkyl groups, C₁-C₄ hydroxyalkyl groups, hydroxyl groups, nitro groups or halo groups (e.g., F, Cl, I, or Br). An aryl group may be ortho-, meta-, and/or para-substituted, preferably para-substituted.

The anion of formula (IV), X⁻, may be any pharmaceutically acceptable anion, for example, Cl⁻, I⁻, Br, ascorbate, hydrofumarate, fumarate, maleate, and the like.

In formula (IV), R₁₀ₐ is taken together with one of R₈, R₈ₐ, R₉, or R₉ₐ to form optionally substituted 3- to 6-membered heterocyclic ring. In some embodiments, R₁₀ₐ may be taken together with one of R₈, R₈ₐ, R₉, or R₉ₐ to form cyclopropyl, cyclobutyl, cyclopentyl, or cyclohexyl.

In formula (IV), R₁ and R₁₂ are for each occurrence independently selected from hydrogen, optionally substituted C₁-C₆ alkyl, optionally substituted C₂-C₆ alkenyl, and optionally substituted aryl. Ru and R₁₂ may each independently be hydrogen. Ru and R₁₂ may each independently be an optionally substituted straight chain or branched C₁-C₆ alkyl, for example a straight chain C₁-C₆ alkyl, or an optionally substituted straight chain or branched C₂-C₆ alkenyl, for example allyl, 2-butenyl, etc. In some embodiments Ru and R₁₂ may each independently be a straight chain or branched C₁-C₄ alkyl, for example a straight chain C₁-C₄ alkyl, or a C₂-C₄ alkenyl. R₁₁ and R₁₂ may each independently be an optionally substituted aryl. An aryl is a 6- to 14-membered aromatic ring, preferably a 6- to 10-membered aromatic ring and includes polycyclic ring systems in which two or more carbon atoms are common to adjoining rings where at least one ring is aromatic. Examples of aryl groups include, but are not limited to phenyl, naphthyl, anthracenyl, and phenantherenyl. An aryl group may be substituted with one or more C₁-C₄ alkyl or perfluoralkyl groups, C₁-C₄ hydroxyalkyl groups, hydroxyl groups, nitro groups or halo groups (e.g., F, Cl, I, or Br). An aryl group may be ortho-, meta-, and/or para-substituted, preferably para-substituted.

Exemplary compounds of formula (IV) are those wherein at least one of R₂, R₃, R₄, or R₅ is selected from hydroxy, —OR₁₁, —OC(O)R₁₁, —OC(O)OR₁₁, —OSO₂R₁₁, and —OC(O)NR₁₁R₁₂.

Other exemplary compounds of formula (IV) are those wherein at least one of R₂, R₃, R₄, or R₅ is selected from hydroxy and —OR₁₁.

Other exemplary compounds of formula (IV) are those wherein at least one of R₂, R₃, R₄, or R₅ is —OR₁₁.

Other exemplary compounds of formula (IV) are those wherein R₁₁ is unsubstituted C₁-C₆ alkyl.

Other exemplary compounds of formula (IV) are those wherein R₁₁ is methyl.

Other exemplary compounds of formula (IV) are those wherein R₃ is methoxy.

Other exemplary compounds of formula (IV) are those wherein R₁₀, is taken together with R₈ to form an optionally substituted 3-membered heterocyclic ring.

Other exemplary compounds of formula (IV) are those wherein R₁₀, is taken together with R₈ to form an optionally substituted 4-membered heterocyclic ring.

Other exemplary compounds of formula (IV) are those wherein R₁₀, is taken together with R₈ to form an optionally substituted 5-membered heterocyclic ring.

Other exemplary compounds of formula (IV) are those wherein R₁₀, is taken together with R₈ to form an optionally substituted 6-membered heterocyclic ring.

Other exemplary compounds of formula (IV) are those wherein R₁₀, is taken together with R₉ to form an optionally substituted 4-membered heterocyclic ring.

Other exemplary compounds of formula (IV) are those wherein R₁₀, is taken together with R₉ to form an optionally substituted 5-membered heterocyclic ring.

Other exemplary compounds of formula (IV) are those wherein R₁₀, is taken together with R₉ to form an optionally substituted 6-membered heterocyclic ring.

Other exemplary compounds of formula (IV) are those wherein the heterocyclic ring is unsubstituted.

Other exemplary compounds of formula (IV) are those wherein R₁₀ is a lone pair.

Other exemplary compounds of formula (IV) are those wherein R₆ is hydrogen.

Other exemplary compounds of formula (IV) are those wherein R₆ₐ is hydrogen.

Other exemplary compounds of formula (IV) are those wherein R₇ is hydrogen.

Other exemplary compounds of formula (IV) are those wherein R₇ₐ is hydrogen.

Other exemplary compounds of formula (IV) are those wherein R₁ is hydrogen.

Exemplary Ibogaine analogs of formula (IV) are shown in Table 4.

TABLE 4
Ibogaine

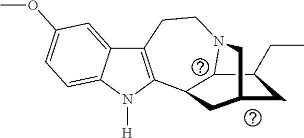

[symbol]

| Exocyclic Series | Endocyclic Series |
|---|---|
| 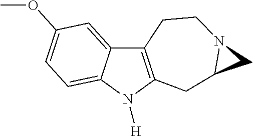 [SMILES: COc1ccc2[nH]c3c(c2c1)CC[N@@]1C[C@H]1C3] (R)-6-methoxy-1,3,4,9,10,10a- [symbol] | 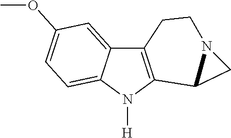 (R)-6-methoxy-3,4,9,9b- [symbol] |
| 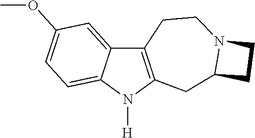 (S)-7-methoxy-1,4,5,10,11,11a- hexahydro-2H- [symbol] | 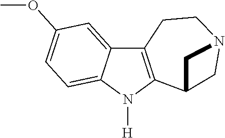 [symbol] |
| 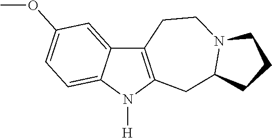 (S)-8-methoxy- 1,2,3,5,6,11,12,12a- [symbol] | 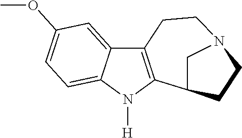 (3R,6R)-10-methoxy-1,2,4,5,6,7-hexahydro-3,6 [symbol] |
| 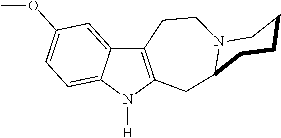 (S)-2-methoxy [symbol] | 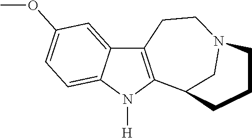 [symbol] |
| 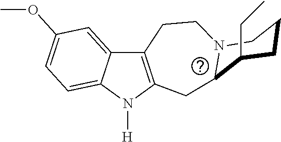 [symbol] | |

[symbol]

indicates data missing or illegible when filed

### Methods of Treatment and Therapeutic Uses

Compounds of formulae (I), (II), (III), or (IV) according to the disclosure, crystalline forms thereof, and the methods and the compositions (e.g., pharmaceutical compositions) are used to regulate the activity of a neurotransmitter receptor by administering a therapeutically effective dose of a compound of formulae (I), (II), (III), or (IV) according to the disclosure, and the methods and the compositions (e.g., pharmaceutical compositions) are used to treat inflammation and/or pain by administering a therapeutically effective dose of a compound of formulae (I), (II), (III), or (IV) according to the disclosure.

Methods of the disclosure also related to the administration of a therapeutically effective amount of a compound of formulae (I), (II), (III), or (IV) according to the disclosure to prevent or treat a disease or condition, such as those discussed below for a subject in need of treatment. Compounds of formulae (I), (II), (III), or (IV) according to the disclosure may be administered neat or as a composition comprising a compound of formulae (I), (II), (III), or (IV) according to the disclosure as discussed below.

Compounds of formulae (I), (II), (III), or (IV) according to the disclosure may be used to prevent and/or treat a psychological disorder. The disclosure provides a method for preventing and/or treating a psychological disorder by administering to a subject in need thereof a therapeutically effective amount of a compound of formulae (I), (II), (III), or (IV) according to the disclosure, including the exemplary embodiments discussed herein. The psychological disorder may be chosen from depression; psychotic disorder; schizophrenia; schizophreniform disorder (acute schizophrenic episode); schizoaffective disorder; bipolar I disorder (mania, manic disorder, manic-depressive psychosis); bipolar II disorder; major depressive disorder; major depressive disorder with psychotic feature (psychotic depression); delusional disorders (paranoia); Shared Psychotic Disorder (Shared paranoia disorder); Brief Psychotic disorder (Other and Unspecified Reactive Psychosis); Psychotic disorder not otherwise specified (Unspecified Psychosis); paranoid personality disorder; schizoid personality disorder; schizotypal personality disorder; anxiety disorder; social anxiety disorder; substance-induced anxiety disorder; selective mutism; panic disorder; panic attacks; agoraphobia; attention deficit syndrome; post-traumatic stress disorder (PTSD); premenstrual dysphoric disorder (PMDD); and premenstrual syndrome (PMS).

Compounds of formulae (I), (II), (III), or (IV) according to the disclosure may be used to treat and/or prevent a substance use disorder. The disclosure provides a method for treating and/or preventing a substance use disorder by administering to a subject in need thereof a therapeutically effective amount of a compound of formulae (I), (II), (III), or (IV) according to the disclosure, including the exemplary embodiments discussed herein. The substance use disorder may be chosen from opioid use disorder; cannabis or marijuana use disorder; nicotine use disorder; stimulant use disorder; sedative use disorder; hypnotic use disorder; anxiolytic use disorder; hallucinogen use disorder; phencyclidine use disorder; inhalant use disorder; caffeine use disorder; and alcohol use disorder.

Compounds of formulae (I), (II), (III), or (IV) according to the disclosure may be used to prevent and/or treat a brain disorder. The disclosure provides a method for preventing and/or treating a brain disorder (e.g., Huntington's disease, Alzheimer's disease, dementia, and Parkinson's disease) by administering to a subject in need thereof a therapeutically effective amount of a compound of formulae (I), (II), (III), or (IV) according to the disclosure, including the exemplary embodiments discussed above.

Compounds of formulae (I), (II), (III), or (IV) according to the disclosure may be used to prevent and/or treat developmental disorders, delirium, dementia, amnestic disorders and other cognitive disorders, psychiatric disorders due to a somatic condition, drug-related disorders, schizophrenia and other psychotic disorders, mood disorders, anxiety disorders, somatoform disorders, factitious disorders, dissociative disorders, eating disorders, sleep disorders, impulse control disorders, adjustment disorders, or personality disorders. The disclosure provides a method for preventing and/or treating these disorders by administering to a subject in need thereof a therapeutically effective amount of a compound of formulae (I), (II), (III), or (IV) according to the disclosure, including the exemplary embodiments discussed above.

Compounds of formulae (I), (II), (III), or (IV) according to the disclosure may be used to prevent and/or treat inflammation and/or pain, such as for example inflammation and/or pain associated with inflammatory skeletal or muscular diseases or conditions. The disclosure provides a method for preventing and/or treating an inflammation and/or pain by administering to a subject in need thereof a therapeutically effective amount of a compound of formulae (I), (II), (III), or (IV) according to the disclosure, including the exemplary embodiments discussed herein. Generally speaking, treatable “pain” includes nociceptive, neuropathic, and mix-type. A method of the disclosure may reduce or alleviate the symptoms associated with inflammation, including but not limited to treating localized manifestation of inflammation characterized by acute or chronic swelling, pain, redness, increased temperature, or loss of function in some cases. A method of the disclosure may reduce or alleviate the symptoms of pain regardless of the cause of the pain, including but not limited to reducing pain of varying severity, i.e., mild, moderate and severe pain, acute pain and chronic pain. A method of the disclosure is effective in treating joint pain, muscle pain, tendon pain, burn pain, and pain caused by inflammation such as rheumatoid arthritis. Skeletal or muscular diseases or conditions which may be treated include but are not limited to musculoskeletal sprains, musculoskeletal strains, tendinopathy, peripheral radiculopathy, osteoarthritis, joint degenerative disease, polymyalgia rheumatica, juvenile arthritis, gout, ankylosing spondylitis, psoriatic arthritis, systemic lupus erythematosus, costochondritis, tendonitis, bursitis, such as the common lateral epicondylitis (tennis elbow), medial epicondylitis (pitchers elbow) and trochanteric bursitis, temporomandibularjoint syndrome, and fibromyalgia.

Compounds of formulae (I), (II), (III), or (IV) according to the disclosure may be used to modulate activity of a mitogen-activated protein kinase (MAPK), comprising administering a composition of the disclosure. MAPKs provide a wide-ranging signaling cascade that allow cells to quickly respond to biotic and abiotic stimuli. Exemplary MAPKs include, but are not limited to, Tropomyosin Receptor Kinase A (TrkA), P38-alpha, and c-Jun N-Terminal Kinase 3 (JNK3). TrkA is a high affinity catalytic receptor of nerve growth factor (NGF) protein. TrkA regulates NGF response, influencing neuronal differentiation and outgrowth as well as programmed cell death. p38-alpha is involved with the regulation of pro-inflammatory cytokines, including TNF-a. In the central nervous system, p38-alpha regulates neuronal death and neurite degeneration, and it is a common target of Alzheimer's disease therapies. JNK3 is neuronal specific protein isoform of the JNKs. It is involved with the regulation of apoptosis. JNK3 also plays a role in modulating the response of cytokines, growth factors, and oxidative stress.

As used herein, the term “modulating activity of a mitogen-activated protein kinase” refers to changing, manipulating, and/or adjusting the activity of a mitogen-activated protein kinase. In one embodiment, modulating the activity of a MAPK can influence neural health, neurogenesis, neural growth and differentiation, and neurodegenerative diseases.

Compounds of formulae (I), (II), (III), or (IV) according to the disclosure may be used to modulate neurogenesis, comprising administering a composition of the disclosure. As used herein, the term “modulating neurogenesis” refers to changing, manipulating, and/or adjusting the growth and development of neural tissue. In one embodiment, neurogenesis comprises adult neurogenesis, in which new neural stem cells are generated from neural stem cells in an adult animal. In one embodiment, modulating neurogenesis comprises increasing and/or enhancing the rate at which new neural tissue is developed.

Compounds of formulae (I), (II), (III), or (IV) according to the disclosure may be used to modulate neurite outgrowth, comprising administering a composition of the disclosure. As used herein, the term “modulating neurite outgrowth” refers to changing, manipulating, and/or adjusting the growth and development of neural projections, or “neurites.” In one embodiment, neurogenesis comprises modulating the growth of new neurites, the number of neurites per neuron, and/or neurite length. In one embodiment, modulating neurite outgrowth comprises increasing and/or enhancing the rate and/or length at which neurites develop.

Compounds of formulae (I), (II), (III), or (IV) according to the disclosure may be used to prevent and/or treat sexual health disorders including, but not limited to, hypoactive sexual desire disorder, hyperactive sexual desire disorder, orgasmic disorder, arousal disorder, vaginismus, and dyspareunia. In some embodiments, the disorder is a male sexual dysfunction disorder. In some embodiments, the disorder is a female sexual dysfunction disorder.

Compounds of formulae (I), (II), (III), or (IV) according to the disclosure may be used to prevent and/or treat women's health disorders including, but not limited to, menstrual cramping, dysmenorrhea, post-hysterectomy pain, vaginal or vulvar vestibule mucosa disorder, menopausal-related disorders, vaginal atrophy, or vulvar vestibulitis.

### Compositions

The disclosure also relates to compositions comprising an effective amount of compounds of formulae (I), (II), (III), or (IV) according to the disclosure, including its exemplary embodiments discussed above, and an excipient (e.g., a pharmaceutically-acceptable excipient). In another embodiment, the disclosure also relates to pharmaceutical compositions comprising a therapeutically effective amount of a compound of formulae (I), (II), (III), or (IV) according to the disclosure, including their exemplary embodiments discussed above, and a pharmaceutically acceptable excipient (also known as a pharmaceutically acceptable carrier). As discussed above, a compound of formulae (I), (II), (III), or (IV) according to the disclosure may be, for example, therapeutically useful to prevent and/or treat the psychological disorders, brain disorders, pain, and inflammation as well as the other disorders described herein.

A composition or a pharmaceutical composition of the disclosure may be in any form which contains a compound of formulae (I), (II), (III), or (IV) according to the disclosure. The composition may be, for example, a tablet, capsule, liquid suspension, injectable, topical, or transdermal. The compositions generally contain, for example, about 1% to about 99% by weight of a compound of formulae (I), (II), (III), or (IV) according to the disclosure and, for example, 99% to 1% by weight of at least one suitable pharmaceutically acceptable excipient. In one embodiment, the composition may be between about 5% and about 75% by weight of a compound of formulae (I), (II), (III), or (IV) according to the disclosure, with the rest being at least one suitable pharmaceutically acceptable excipient or at least one other adjuvant, as discussed below.

Published US applications US 2018/0221396 A1 and US 2019/0142851 A1 disclose compositions comprising a combination of a first purified psilocybin derivative with a second purified psilocybin derivative, with one or two purified cannabinoids or with a purified terpene. Various ratios of these components in the composition are also disclosed. The disclosures of US 2018/0221396 A1 and US 2019/0142851 A1 are incorporated herein by reference. According to this disclosure, a compound of formulae (I), (II), (III), or (IV) according to the disclosure may be used as the “first purified psilocybin derivative” in the compositions described in US 2018/0221396 A1 and US 2019/0142851 A1.

Accordingly, this disclosure provides a composition comprising: a first component comprising at least one compound of formulae (I), (II), (III), or (IV) according to the disclosure; at least one second component selected from at least one of (a) a serotonergic drug, (b) a purified psilocybin derivative, (c) a purified cannabinoid or (d) a purified terpene; and at least one pharmaceutically-acceptable excipient or at least one other adjuvant. Such a composition may be a pharmaceutical composition wherein the components are present individually in therapeutically effective amounts or by combination in a therapeutically effective amount to treat a disease, disorder, or condition as described herein.

When used in such compositions as a first component comprising at least one compound of formulae (I), (II), (III), or (IV) according to the disclosure with a second component selected from at least one of (a) a serotonergic drug, (b) a purified psilocybin derivative, (c) a purified cannabinoid, or (d) a purified terpene, the compositions represent particular embodiments of the disclosure. Compositions having as a first component at least one compound of formulae (I), (II), (III), or (IV) according to the disclosure with a second component selected from at least one of (e) an adrenergic drug, (f) a dopaminergic drug, (g) a monoamine oxidase inhibitor, (h) a purified erinacine, or (i) a purified hericenone, also represent additional particular embodiments of the disclosure represented by the compositions having the compound of formulae (I), (II), (III), or (IV) according to the disclosure. In some embodiments, the first and second components can be administered at the same time (e.g., together in the same composition), or at separate times over the course of treating a patient in need thereof. Such a composition may be a pharmaceutical composition wherein the components are present individually in therapeutically effective amounts or by combination in a therapeutically effective amount to treat a disease, disorder, or condition as described herein.

Within the context of this disclosure, the term “purified” means separated from other materials, such as plant or fungal material, e.g., protein, chitin, cellulose, or water. In one embodiment, the term “purified” refers to a compound substantially free of other materials. In one embodiment, the term “purified” refers to a compound that is substantially free from a second tryptamine compound. In one embodiment, the term “purified” refers to a compound substantially free from histidine. In one embodiment, the term “purified” refers to a compound substantially free from a biological material, such as mold, fungus, plant matter, or bacteria. In one embodiment, the term “purified” refers to a compound substantially free from a paralytic.

In one embodiment, the term “purified” refers to a compound which has been separated from other compounds that are typically co-extracted when the purified compound is extracted from a naturally occurring organism. In one embodiment, a “purified” psilocybin derivative is partially or completely isolated from other psilocybin derivatives present in a source material, such as a psilocybin-containing mushroom. In one example, “purified” baeocystin is substantially free from psilocybin and/or psilocin. By contrast, traditional psilocybin mushroom extracts (aka crude extracts or fruit body extracts) would be expected to contain an unpredictable and varying amount of psilocybin, psilocin, baeocystin, norbaeocystin, salts thereof, or combinations thereof. Other examples of unpurified psilocybin derivatives would include mycelium containing psilocybin derivatives and/or naturally occurring fungal material such as biological material and/or structural material such as chitin. Similarly, the term “cannabis extracts” or “cannabinoid extracts” traditionally refers to whole plants (aka crude or full spectrum extracts) which have not been subjected to further purification to eliminate unwanted molecules that naturally occur in the cannabis plant. For example, a “cannabis extract comprising cannabidiol” could be expected to include cannabidiol (aka “CBD”) and also varying amounts of other compounds, including cannabinoids, terpenes, and other biological material.

In one embodiment, the term “purified” refers to a compound or composition that has been crystallized.

In one embodiment, the term “purified” refers to a compound or composition that has been chromatographed, for example by gas chromatography, liquid chromatography (e.g., LC, HPLC, etc.), etc.

In one embodiment, the term “purified” refers to a compound or composition that has been distilled.

In one embodiment, the term “purified” refers to a compound or composition that has been sublimed.

In one embodiment, the term “purified” refers to a compound or composition that has been subject to two or more steps chosen from crystallization, chromatography, distillation, or sublimation.

In one embodiment, the term “purified” refers to a compound that is between 80-100% pure.

In one embodiment, the term “purified” refers to a compound that is between 90-100% pure.

In one embodiment, the term “purified” refers to a compound that is between 95-100% pure.

In one embodiment, the term “purified” refers to a compound that is between 99-100% pure.

In one embodiment, the term “purified” refers to a compound that is between 99.9-100% pure.

A serotonergic drug refers to a compound that binds to, blocks, or otherwise influences (e.g., via an allosteric reaction) activity at a serotonin receptor as described in paragraphs [0245]-[0253] of US 2018/0221396 A1 and [0305]-[0311] US 2019/0142851 A1 as well as the disclosed exemplary embodiments, incorporated here by reference. Exemplary psilocybin derivatives include but are not limited to psilocybin itself and the psilocybin derivates described in paragraphs [0081]-[0109] of US 2018/0221396 A1 and [0082]-[0110] US 2019/0142851 A1 as well as the disclosed exemplary embodiments. Exemplary cannabinoids include but are not limited to the cannabinoids described in paragraphs [0111]-[0145] of US 2018/0221396 A1 and [0112]-[0146] US 2019/0142851 A1 as well as the disclosed exemplary embodiments. Exemplary terpenes include but are not limited to the terpenes described in paragraphs [0160]-[0238] of US 2018/0221396 A1 and [0161]-[0300] US 2019/0142851 A1 as well as the disclosed exemplary embodiments.

A pharmaceutical formulation of the disclosure may comprise, consist essentially of, or consist of (a) at least one compound of formulae (I), (II), (III), or (IV) according to the disclosure and (b) at least one second active compound selected from a serotonergic drug, a purified psilocybin derivative, a purified cannabinoid, a purified terpene, an adrenergic drug, a dopaminergic drug, a monoamine oxidase inhibitor, a purified erinacine, or a purified hericenone and (c) a pharmaceutically acceptable excipient. In some embodiments, the compound(s) of formulae (I), (II), (III), or (IV) according to the disclosure and the second active compound(s) are each present in a therapeutically effective amount using a purposefully engineered and unnaturally occurring molar ratios. Exemplary molar ratios of the compounds of formulae (I), (II), (III), or (IV) according to the disclosure to the second active compound in a composition of the disclosure include but are not limited to from about 0.1:100 to about 100:0.1, from about 1:100 to about 100:1, from about 1:50 to about 50:1, from about 1:25 to about 25:1, from about 1:20 to about 20:1, from about 1:10 to about 10:1, from about 1:5 to about 5:1, from about 1:2 to about 2:1 or may be about 1:1.

A pharmaceutical formulation of the disclosure may comprise a composition containing a compound of formulae (I), (II), (III), or (IV) according to the disclosure and a serotonergic drug, a purified psilocybin derivative, a purified cannabinoid, or a purified terpene, each present in a therapeutically effective amount using a purposefully engineered and unnaturally occurring molar ratios. Published US applications US 2018/0221396 A1 and US 2019/0142851 A1 disclose compositions comprising a combination of a purified psilocybin derivative with a second purified psilocybin derivative, with one or two purified cannabinoids or with a purified terpene. According to this disclosure composition containing a compound of formulae (I), (II), (III), or (IV) according to the disclosure may be used in place of a “purified psilocybin derivative” in the compositions described in US 2018/0221396 A1 and US 2019/0142851 A1. Accordingly, the disclosure provides a pharmaceutical formulation comprising as (a) at least one compound of formulae (I), (II), (III), or (IV) according to the disclosure and at least one second component selected from (b) a purified psilocybin derivative, (c) a purified cannabinoid or (d) a purified terpene; and at least one pharmaceutically-acceptable excipient or at least one other adjuvant, as described herein. Such a composition may be a pharmaceutical composition wherein the components are present individually in therapeutically effective amounts or by combination in a therapeutically effective amount to treat a disease, disorder, or condition as described herein.

A serotonergic drug refers to a compound that binds to, blocks, or otherwise influences (e.g., via an allosteric reaction) activity at a serotonin receptor as described in paragraphs [0245]-[0253] of US 2018/0221396 A1 and [0305]-[0311] US 2019/0142851 A1 as well as the disclosed exemplary embodiments. Some exemplary serotonergic drugs include SSRIs and SNRIs. Some examples of specific serotonergic drugs include the following molecules, including any salts, solvates, or polymorphs thereof: 6-allyl-N,N-diethyl-NL; N,N-dibutyl-T; N,N-diethyl-T; N,N-diisopropyl-T; 5-methyoxy-alpha-methyl-T; N,N-dimethyl-T; 2,alpha-dimethyl-T; alpha,N-dimethyl-T; N,N-dipropyl-T; N-ethyl-N-isopropyl-T; alpha-ethyl-T; 6-N,N-triethyl-NL; 3,4-dihydro-7-methoxy-1-methyl-C; 7-methyoxy-1-methyl-C; N,N-dibutyl-4-hydroxy-T; N,N-diethyl-4-hydroxy-T; N,N-diisopropyl-4-hydroxy-T; N,N-dimethyl-4-hydroxy-T; N,N-dimethyl-5-hydroxy-T; N,N-dipropyl-4-hydroxy-T; N-ethyl-4-hydroxy-N-methyl-T; 4-hydroxy-N-isopropyl-N-methyl-T; 4-hydroxy-N-methyl-N-propyl-T; 4-hydroxy-N,N-tetramethylene-T; ibogaine; N,N-diethyl-L; N-butyl-N-methyl-T; N,N-diisopropyl-4,5-methylenedioxy-T; N,N-diisopropyl-5,6-methylenedioxy-T; N,N-dimethyl-4,5-methylenedioxy-T; N,N-dimethyl-5,6-methylenedioxy-T; N-isopropyl-N-methyl-5,6-methylenedioxy-T; N,N-diethyl-2-methyl-T; 2-N,N-trimethyl-T; N-acetyl-5-methoxy-T; N,N-diethyl-5-methoxy-T; N,N-diisopropyl-5-methoxy-T; 5-methoxy-N,N-dimethyl-T; N-isopropyl-4-methoxy-N-methyl-T; N-isopropyl-5-methoxy-N-methyl-T; 5,6-dimethoxy-N-isopropyl-N-methyl-T; 5-methoxy-N-methyl-T; 5-methoxy-N,N-tetramethylene-T; 6-methoxy-1-methyl-1,2,3,4-tetrahydro-C; 5-methoxy-2-N,N-trimethyl-T; N,N-dimethyl-5-methylthio-T; N-isopropyl-N-methyl-T; alpha-methyl-T; N-ethyl-T; N-methyl-T; 6-propyl-NL; N,N-tetramethylene-T; tryptamine; 7-methoxy-1-methyl-1,2,3,4-tetrahydro-C; and alpha,N-dimethyl-5-methoxy-T. For additional information regarding these compounds see Shulgin, A. T., & Shulgin, A. (2016). Tihkal: The Continuation. Berkeley, Calif.: Transform Press. In one embodiment, a serotonergic drug is chosen from alprazolam, amphetamine, aripiprazole, azapirone, a barbiturate, bromazepam, bupropion, buspirone, a cannabinoid, chlordiazepoxide, citalopram, clonazepam, clorazepate, dextromethorphan, diazepam, duloxetine, escitalopram, fluoxetine, flurazepam, fluvoxamine, lorazepam, lysergic acid diethylamide, lysergamide, 3,4-methylenedioxymethamphetamine, milnacipran, mirtazapine, naratriptan, paroxetine, pethidine, phenethylamine, psicaine, oxazepam, reboxetine, serenic, serotonin, sertraline, temazepam, tramadol, triazolam, a tryptamine, venlafaxine, vortioxetine, and/or derivatives thereof. In an exemplary embodiment, the serotonergic drug is 3,4-methylenedioxymethamphetamine.

Exemplary psilocybin derivatives include but are not limited to psilocybin itself and the psilocybin derivates described in paragraphs [0081]-[0109] of US 2018/0221396 A1 and [0082]-[0110] US 2019/0142851 A1 as well as the disclosed exemplary embodiments. In one embodiment, the compositions disclosed herein comprise one or more purified psilocybin derivatives chosen from: [3-(2-dimethylaminoethyl)-1H-indol-4-yl]dihydrogen phosphate; 4-hydroxytryptamine; 4-hydroxy-N,N-dimethyltryptamine; [3-(2-methylaminoethyl)-1H-indol-4-yl]dihydrogen phosphate; 4-hydroxy-N-methyltryptamine; [3-(aminoethyl)-1H-indol-4-yl]dihydrogen phosphate; [3-(2-trimethylaminoethyl)-1H-indol-4-yl]dihydrogen phosphate; and 4-hydroxy-N,N,N-trimethyltryptamine.

Exemplary cannabinoids include but are not limited to the cannabinoids described in paragraphs [0111]-[0145] of US 2018/0221396 A1 and [0112]-[0146] US 2019/0142851 A1 as well as the disclosed exemplary embodiments, incorporated here by reference. Examples of cannabinoids within the context of this disclosure include the following molecules: cannabichromene (CBC); cannabichromenic acid (CBCA); cannabichromevarin (CBCV); cannabichromevarinic acid (CBCVA); cannabicyclol (CBL); cannabicyclolic acid (CBLA); cannabicyclovarin (CBLV); cannabidiol (CBD); cannabidiol monomethylether (CBDM); cannabidiolic acid (CBDA); cannabidiorcol (CBD-C1); cannabidivarin (CBDV); cannabidivarinic acid (CBDVA); cannabielsoic acid B (CBEA-B); cannabielsoin (CBE); cannabielsoin acid A (CBEA-A); cannabigerol (CBG); cannabigerol monomethylether (CBGM); cannabigerolic acid (CBGA); cannabigerolic acid monomethylether (CBGAM); cannabigerovarin (CBGV); cannabigerovarinic acid (CBGVA); cannabinodiol (CBND); cannabinodivarin (CBVD); cannabinol (CBN); cannabinol methylether (CBNM); cannabinol-C2 (CBN-C2); cannabinol-C4 (CBN-C4); cannabinolic acid (CBNA); cannabiorcol (CBN-C1); cannabivarin (CBV); cannabitriol (CBT); cannabitriolvarin (CBTV); 10-ethoxy-9-hydroxy-delta-6a-tetrahydrocannabinol; cannabicitran (CBTC); cannabiripsol (CBR); 8,9-dihydroxy-delta-6a-tetrahydrocannabinol; delta-8-tetrahydrocannabinol (A8-THC); delta-8-tetrahydrocannabinolic acid (A8-THCA); delta-9-tetrahydrocannabinol (THC); delta-9-tetrahydrocannabinol-C4 (THC-C4); delta-9-tetrahydrocannabinolic acid A (THCA-A); delta-9-tetrahydrocannabinolic acid B (THCA-B); delta-9-tetrahydrocannabinolic acid-C4 (THCA-C4); delta-9-tetrahydrocannabiorcol (THC-C1); delta-9-tetrahydrocannabiorcolic acid (THCA-C1); delta-9-tetrahydrocannabivarin (THCV); delta-9-tetrahydrocannabivarinic acid (THCVA); 10-oxo-delta-6a-tetrahydrocannabinol (OTHC); cannabichromanon (CBCF); cannabifuran (CBF); cannabiglendol; delta-9-cis-tetrahydrocannabinol (cis-THC); trihydroxy-delta-9-tetrahydrocannabinol (triOH-THC); dehydrocannabifuran (DCBF); and 3,4,5,6-tetrahydro-7-hydroxy-alpha-alpha-2-trimethyl-9-n-propyl-2,6-methano-2H-1-benzoxocin-5-methanol. In one embodiment, the purified cannabinoid is chosen from THC, THCA, THCV, THCVA, CBC, CBCA, CBCV, CBCVA, CBD, CBDA, CBDV, CBDVA, CBG, CBGA, CBGV, or CBGVA.

Exemplary terpenes include but are not limited to the terpenes described in paragraphs [0160]-[0238] of US 2018/0221396 A1 and [0161]-[0300] US 2019/0142851 A1 as well as the disclosed exemplary embodiments. In one embodiment, a purified terpene is chosen from acetanisole, acetyl cedrene, anethole, anisole, benzaldehyde, bornyl acetate, borneol, cadinene, cafestol, caffeic acid, camphene, camphor, capsaicin, carene, carotene, carvacrol, carvone, caryophyllene, caryophyllene, caryophyllene oxide, cedrene, cedrene epoxide, cecanal, cedrol, cembrene, cinnamaldehyde, cinnamic acid, citronellal, citronellol, cymene, eicosane, elemene, estragole, ethyl acetate, ethyl cinnamate, ethyl maltol, eucalyptol/1,8-cineole, eudesmol, eugenol, euphol, farnesene, farnesol, fenchone, geraniol, geranyl acetate, guaia-1(10),11-diene, guaiacol, guaiol, guaiene, gurjunene, herniarin, hexanaldehyde, hexanoic acid, humulene, ionone, ipsdienol, isoamyl acetate, isoamyl alcohol, isoamyl formate, isoborneol, isomyrcenol, isoprene, isopulegol, isovaleric acid, lavandulol, limonene, gamma-linolenic acid, linalool, longifolene, lycopene, menthol, methyl butyrate, 3-mercapto-2-methylpentanal, beta-mercaptoethanol, mercaptoacetic acid, methyl salicylate, methylbutenol, methyl-2-methylvalerate, methyl thiobutyrate, myrcene, gamma-muurolene, nepetalactone, nerol, nerolidol, neryl acetate, nonanaldehyde, nonanoic acid, ocimene, octanal, octanoic acid, pentyl butyrate, phellandrene, phenylacetaldehyde, phenylacetic acid, phenylethanethiol, phytol, pinene, propanethiol, pristimerin, pulegone, retinol, rutin, sabinene, squalene, taxadiene, terpineol, terpine-4-ol, terpinolene, thujone, thymol, umbelliferone, undecanal, verdoxan, or vanillin. In one embodiment, a purified terpene is chosen from bornyl acetate, alpha-bisabolol, borneol, camphene, camphor, carene, caryophyllene, cedrene, cymene, elemene, eucalyptol, eudesmol, farnesene, fenchol, geraniol, guaiacol, humulene, isoborneol, limonene, linalool, menthol, myrcene, nerolidol, ocimene, phellandrene, phytol, pinene, pulegone, sabinene, terpineol, terpinolene, or valencene.

As used herein, the term “adrenergic drug” refers to a compound that binds, blocks, or otherwise influences (e.g., via an allosteric reaction) activity at an adrenergic receptor. In one embodiment, an adrenergic drug binds to an adrenergic receptor. In one embodiment, an adrenergic drug indirectly affects an adrenergic receptor, e.g., via interactions affecting the reactivity of other molecules at the adrenergic receptor. In one embodiment, an adrenergic drug is an agonist, e.g., a compound activating an adrenergic receptor. In one embodiment, an adrenergic drug is an antagonist, e.g., a compound binding but not activating an adrenergic receptor, e.g., blocking a receptor. In one embodiment, an adrenergic drug is an effector molecule, e.g., a compound binding to an enzyme for allosteric regulation. In one embodiment, an adrenergic drug acts (either directly or indirectly) at more than one type of receptor (e.g., 5HT, dopamine, adrenergic, acetylcholine, etc.).

In one embodiment, an adrenergic drug is an antidepressant. In one embodiment, an adrenergic drug is a norepinephrine transporter inhibitor. In one embodiment, an adrenergic drug is a vesicular monoamine transporter inhibitor. In one embodiment, an adrenergic drug is chosen from adrenaline, agmatine, amoxapine, aptazapine, atomoxetine, bupropion, clonidine, doxepin, duloxetine, esmirtazpine, mianserin, ketanserin, mirabegron, mirtazapine, norepinephrine, phentolamine, phenylephrine, piperoxan, reserpine, ritodrine, setiptiline, tesofensine, timolol, trazodone, trimipramine, or xylazine.

As used herein, the term “dopaminergic drug” refers to a compound that binds, blocks, or otherwise influences (e.g., via an allosteric reaction) activity at a dopamine receptor. In one embodiment, a dopaminergic drug binds to a dopamine receptor. In one embodiment, a dopaminergic drug indirectly affects a dopamine receptor, e.g., via interactions affecting the reactivity of other molecules at the dopamine receptor. In one embodiment, a dopaminergic drug is an agonist, e.g., a compound activating a dopamine receptor. In one embodiment, a dopaminergic drug is an antagonist, e.g., a compound binding but not activating a dopamine receptor, e.g., blocking a receptor. In one embodiment, a dopaminergic drug is an effector molecule, e.g., a compound binding to an enzyme for allosteric regulation. In one embodiment, a dopaminergic drug acts (either directly or indirectly) at more than one type of receptor (e.g., 5HT, dopamine, adrenergic, acetylcholine, etc.).

In one embodiment, a dopaminergic drug is a dopamine transporter inhibitor. In one embodiment, a dopaminergic drug is a vesicular monoamine transporter inhibitor. In one embodiment, a dopaminergic drug is chosen from amineptine, apomorphine, benzylpiperazine, bromocriptine, cabergoline, chlorpromazine, clozapine, dihydrexidine, domperidone, dopamine, fluphenazine, haloperidol, ketamine, loxapine, methamphetamine, olanzapine, pemoline, perphenazine, pergolide, phencyclidine, phenethylamine, phenmetrazine, pimozide, piribedil, a psychostimulant, reserpine, risperidone, ropinirole, tetrabenazine, or thioridazine.

As used herein, the term “monoamine oxidase inhibitor” (MAOI) refers to a compound that blocks the actions of monoamine oxidase enzymes. In one embodiment, a MAOI inhibits the activity of one or both monoamine oxidase A and monoamine oxidase B. In one embodiment a MAOI is a reversible inhibitors of monoamine oxidase A. In one embodiment a MAOI is a drug chosen from isocarboxazid, phenelzine, or tranylcypromine. In one embodiment, a MAOI is β-carboline, pinoline, harmane, harmine, harmaline, harmalol, tetrahydroharmine, 9-methyl-β-carboline, or 3-carboxy-tetrahydrononharman.

In one embodiment, the compositions and methods disclosed herein include one or more purified erinacine molecules. In one embodiment, the compositions and methods disclosed herein comprise purified erinacine A. In one embodiment, the compositions and methods disclosed herein comprise erinacine B. In one embodiment, the compositions and methods disclosed herein comprise erinacine C. In one embodiment, the compositions and methods disclosed herein comprise erinacine D.

In one embodiment, the compositions and methods disclosed herein comprise erinacine E. In one embodiment, the compositions and methods disclosed herein comprise erinacine F. In one embodiment, the compositions and methods disclosed herein comprise erinacine G. In one embodiment, the compositions and methods disclosed herein comprise erinacine H. In one embodiment, the compositions and methods disclosed herein comprise erinacine I. In one embodiment, the compositions and methods disclosed herein comprise erinacine J. In one embodiment, the compositions and methods disclosed herein comprise erinacine K In one embodiment, the compositions and methods disclosed herein comprise erinacine P. In one embodiment, the compositions and methods disclosed herein comprise erinacine Q. In one embodiment, the compositions and methods disclosed herein comprise erinacine R. In one embodiment, the compositions and methods disclosed herein comprise erinacine S.

In one embodiment, the compositions and methods disclosed herein include one or more purified hericenone molecules. In one embodiment, the compositions and methods disclosed herein comprise purified hericenone A. In one embodiment, the compositions and methods disclosed herein comprise purified hericenone B. In one embodiment, the compositions and methods disclosed herein comprise purified hericenone C. In one embodiment, the compositions and methods disclosed herein comprise purified hericenone D. In one embodiment, the compositions and methods disclosed herein comprise purified hericenone E. In one embodiment, the compositions and methods disclosed herein comprise purified hericenone F. In one embodiment, the compositions and methods disclosed herein comprise purified hericenone G. In one embodiment, the compositions and methods disclosed herein comprise purified hericenone H.

Exemplary compositions of a compound of formulae (I), (II), (III), or (IV) according to the disclosure and a second compound selected from a serotonergic drug, a purified psilocybin derivative, a purified cannabinoid, a purified terpene, an adrenergic drug, a dopaminergic drug, a monoamine oxidase inhibitor, a purified erinacine, or a purified hericenone in exemplary molar ratios are shown in Table 5. A compound of formulae (I), (II), (III), or (IV) according to the disclosure may be any one of the exemplary embodiments described above including their crystalline forms as disclosed herein.

TABLE 5

| | Molar ratio of a | Molar ratio of a | Molar ratio of a |
|---|---|---|---|
| | compound of | compound of | compound of |
| | formulae (I), (II), | formulae (I), (II), | formulae (I), (II), |
| | (III), or (IV):second | (III), or (IV):second | (III), or (IV):second |
| Second Compound | compound | compound | compound |
| 3,4- | About 1:100 to | About 1:25 to | About 1:5 to |
| methylenedioxymethamphetamine | about 100:1 | about 25:1 | about 5:1 |
| Citalopram | About 1:100 to | About 1:25 to | About 1:5 to |
| | about 100:1 | about 25:1 | about 5:1 |
| Escitalopram | About 1:100 to | About 1:25 to | About 1:5 to |
| | about 100:1 | about 25:1 | about 5:1 |
| Fluoxetine | About 1:100 to | About 1:25 to | About 1:5 to |
| | about 100:1 | about 25:1 | about 5:1 |
| Paroxetine | About 1:100 to | About 1:25 to | About 1:5 to |
| | about 100:1 | about 25:1 | about 5:1 |
| Sertraline | About 1:100 to | About 1:25 to | About 1:5 to |
| | about 100:1 | about 25:1 | about 5:1 |
| Duloxetine | About 1:100 to | About 1:25 to | About 1:5 to |
| | about 100:1 | about 25:1 | about 5:1 |
| [3-(2-dimethylaminoethyl)-1H- | About 1:100 to | About 1:25 to | About 1:5 to |
| indol-4-yl] dihydrogen phosphate | about 100:1 | about 25:1 | about 5:1 |
| 4-hydroxytryptamine | About 1:100 to | About 1:25 to | About 1:5 to |
| | about 100:1 | about 25:1 | about 5:1 |
| 4-hydroxy-N,N-dimethyltryptamine | About 1:100 to | About 1:25 to | About 1:5 to |
| | about 100:1 | about 25:1 | about 5:1 |
| [3-(2-methylaminoethyl)-1H-indol- | About 1:100 to | About 1:25 to | About 1:5 to |
| 4-yl] dihydrogen phosphate | about 100:1 | about 25:1 | about 5:1 |
| 4-hydroxy-N-methyltryptamine | About 1:100 to | About 1:25 to | About 1:5 to |
| | about 100:1 | about 25:1 | about 5:1 |
| [3-(aminoethyl)-1H-indol-4-yl] | About 1:100 to | About 1:25 to | About 1:5 to |
| dihydrogen phosphate | about 100:1 | about 25:1 | about 5:1 |
| [3-(2-trimethylaminoethyl)-1H- | About 1:100 to | About 1:25 to | About 1:5 to |
| indol-4-yl] dihydrogen phosphate | about 100:1 | about 25:1 | about 5:1 |
| 4-hydroxy-N,N,N- | About 1:100 to | About 1:25 to | About 1:5 to |
| trimethyltryptamine | about 100:1 | about 25:1 | about 5:1 |
| THC | About 1:100 to | About 1:25 to | About 1:5 to |
| | about 100:1 | about 25:1 | about 5:1 |
| CBC | About 1:100 to | About 1:25 to | About 1:5 to |
| | about 100:1 | about 25:1 | about 5:1 |
| CBD | About 1:100 to | About 1:25 to | About 1:5 to |
| | about 100:1 | about 25:1 | about 5:1 |
| CBG | About 1:100 to | About 1:25 to | About 1:5 to |
| | about 100:1 | about 25:1 | about 5:1 |
| Myrcene | About 1:100 to | About 1:25 to | About 1:5 to |
| | about 100:1 | about 25:1 | about 5:1 |
| Pinene | About 1:100 to | About 1:25 to | About 1:5 to |
| | about 100:1 | about 25:1 | about 5:1 |
| Caryophyllene | About 1:100 to | About 1:25 to | About 1:5 to |
| | about 100:1 | about 25:1 | about 5:1 |
| Limonene | About 1:100 to | About 1:25 to | About 1:5 to |
| | about 100:1 | about 25:1 | about 5:1 |
| Humulene | About 1:100 to | About 1:25 to | About 1:5 to |
| | about 100:1 | about 25:1 | about 5:1 |
| Linalool | About 1:100 to | About 1:25 to | About 1:5 to |
| | about 100:1 | about 25:1 | about 5:1 |
| Adrenaline | About 1:100 to | About 1:25 to | About 1:5 to |
| | about 100:1 | about 25:1 | about 5:1 |
| Amineptine | About 1:100 to | About 1:25 to | About 1:5 to |
| | about 100:1 | about 25:1 | about 5:1 |
| Erinacine A | About 1:100 to | About 1:25 to | About 1:5 to |
| | about 100:1 | about 25:1 | about 5:1 |
| Hericenone A | About 1:100 to | About 1:25 to | About 1:5 to |
| | about 100:1 | about 25:1 | about 5:1 |
| Phenelzine | About 1:100 to | About 1:25 to | About 1:5 to |
| | about 100:1 | about 25:1 | about 5:1 |

Exemplary pharmaceutical compositions of a compound of formulae (I), (II), (III), or (IV) according to the disclosure and a second compound selected from a serotonergic drug, a purified psilocybin derivative, a purified cannabinoid, a purified terpene, an adrenergic drug, a dopaminergic drug, a monoamine oxidase inhibitor, a purified erinacine, and a purified hericenone and an excipient with exemplary molar ratios of a compound of formulae (I), (II), (III), or (IV) according to the disclosure to the second compound are shown in Table 6. A compound of formulae (I), (II), (III), or (IV) according to the disclosure may be any one of the exemplary embodiments described above including their crystalline forms as disclosed herein.

TABLE 6

| | Molar ratio of a | Molar ratio of a | Molar ratio of a |
|---|---|---|---|
| | compound of | compound of | compound of |
| | formulae (I), (II), | formulae (I), (II), | formulae (I), (II), |
| | (III), or (IV):second | (III), or (IV):second | (III), or (IV):second |
| Second Compound | compound | compound | compound |
| 3,4- | About 1:100 to | About 1:25 to | About 1:5 to |
| methylenedioxymethamphetamine | about 100:1 | about 25:1 | about 5:1 |
| Citalopram | About 1:100 to | About 1:25 to | About 1:5 to |
| | about 100:1 | about 25:1 | about 5:1 |
| Escitalopram | About 1:100 to | About 1:25 to | About 1:5 to |
| | about 100:1 | about 25:1 | about 5:1 |
| Fluoxetine | About 1:100 to | About 1:25 to | About 1:5 to |
| | about 100:1 | about 25:1 | about 5:1 |
| Paroxetine | About 1:100 to | About 1:25 to | About 1:5 to |
| | about 100:1 | about 25:1 | about 5:1 |
| Sertraline | About 1:100 to | About 1:25 to | About 1:5 to |
| | about 100:1 | about 25:1 | about 5:1 |
| Duloxetine | About 1:100 to | About 1:25 to | About 1:5 to |
| | about 100:1 | about 25:1 | about 5:1 |
| [3-(2-dimethylaminoethyl)-1H- | About 1:100 to | About 1:25 to | About 1:5 to |
| indol-4-yl] dihydrogen phosphate | about 100:1 | about 25:1 | about 5:1 |
| 4-hydroxytryptamine | About 1:100 to | About 1:25 to | About 1:5 to |
| | about 100:1 | about 25:1 | about 5:1 |
| 4-hydroxy-N,N-dimethyltryptamine | About 1:100 to | About 1:25 to | About 1:5 to |
| | about 100:1 | about 25:1 | about 5:1 |
| [3-(2-methylaminoethyl)-1H-indol- | About 1:100 to | About 1:25 to | About 1:5 to |
| 4-yl] dihydrogen phosphate | about 100:1 | about 25:1 | about 5:1 |
| 4-hydroxy-N-methyltryptamine | About 1:100 to | About 1:25 to | About 1:5 to |
| | about 100:1 | about 25:1 | about 5:1 |
| [3-(aminoethyl)-1H-indol-4-yl] | About 1:100 to | About 1:25 to | About 1:5 to |
| dihydrogen phosphate | about 100:1 | about 25:1 | about 5:1 |
| [3-(2-trimethylaminoethyl)-1H- | About 1:100 to | About 1:25 to | About 1:5 to |
| indol-4-yl] dihydrogen phosphate | about 100:1 | about 25:1 | about 5:1 |
| 4-hydroxy-N,N,N- | About 1:100 to | About 1:25 to | About 1:5 to |
| trimethyltryptamine | about 100:1 | about 25:1 | about 5:1 |
| THC | About 1:100 to | About 1:25 to | About 1:5 to |
| | about 100:1 | about 25:1 | about 5:1 |
| CBC | About 1:100 to | About 1:25 to | About 1:5 to |
| | about 100:1 | about 25:1 | about 5:1 |
| CBD | About 1:100 to | About 1:25 to | About 1:5 to |
| | about 100:1 | about 25:1 | about 5:1 |
| CBG | About 1:100 to | About 1:25 to | About 1:5 to |
| | about 100:1 | about 25:1 | about 5:1 |
| Myrcene | About 1:100 to | About 1:25 to | About 1:5 to |
| | about 100:1 | about 25:1 | about 5:1 |
| Pinene | About 1:100 to | About 1:25 to | About 1:5 to |
| | about 100:1 | about 25:1 | about 5:1 |
| Caryophyllene | About 1:100 to | About 1:25 to | About 1:5 to |
| | about 100:1 | about 25:1 | about 5:1 |
| Limonene | About 1:100 to | About 1:25 to | About 1:5 to |
| | about 100:1 | about 25:1 | about 5:1 |
| Humulene | About 1:100 to | About 1:25 to | About 1:5 to |
| | about 100:1 | about 25:1 | about 5:1 |
| Linalool | About 1:100 to | About 1:25 to | About 1:5 to |
| | about 100:1 | about 25:1 | about 5:1 |
| Adrenaline | About 1:100 to | About 1:25 to | About 1:5 to |
| | about 100:1 | about 25:1 | about 5:1 |
| Amineptine | About 1:100 to | About 1:25 to | About 1:5 to |
| | about 100:1 | about 25:1 | about 5:1 |
| Erinacine A | About 1:100 to | About 1:25 to | About 1:5 to |
| | about 100:1 | about 25:1 | about 5:1 |
| Hericenone A | About 1:100 to | About 1:25 to | About 1:5 to |
| | about 100:1 | about 25:1 | about 5:1 |
| Phenelzine | About 1:100 to | About 1:25 to | About 1:5 to |
| | about 100:1 | about 25:1 | about 5:1 |

An “effective amount” or a “therapeutically effective amount” of a compound of formulae (I), (II), (III), or (IV) according to the disclosure is generally in the range of about 0.1 to about 100 mg daily (oral dose), of about 0.1 to about 50 mg daily (oral dose) of about 0.25 to about 25 mg daily (oral dose), of about 0.1 to about 5 mg daily (oral dose) or of about 0.5 to about 2.5 mg daily (oral dose). The actual amount required for treatment of any particular patient may depend upon a variety of factors including, for example, the disease being treated and its severity; the specific pharmaceutical composition employed; the age, body weight, general health, sex, and diet of the patient; the mode of administration; the time of administration; the route of administration; and the rate of excretion; the duration of the treatment; any drugs used in combination or coincidental with the specific compound employed; and other such factors well known in the medical arts. These factors are discussed in Goodman and Gilman's “The Pharmacological Basis of Therapeutics,” Tenth Edition, A. Gilman, J. Hardman and L. Limbird, eds., McGraw-Hill Press, 155-173 (2001), which is incorporated herein by reference. A compound of formulae (I), (II), (III), or (IV) according to the disclosure and pharmaceutical compositions containing it may be used in combination with other agents that are generally administered to a patient being treated for psychological and other disorders discussed above. They may also be co-formulated with one or more of such agents in a single pharmaceutical composition.

Depending on the type of pharmaceutical composition, the pharmaceutically acceptable carrier may be chosen from any one or a combination of carriers known in the art. The choice of the pharmaceutically acceptable carrier depends upon the pharmaceutical form and the desired method of administration to be used. Exemplary carriers include those that do not substantially alter the structure or activity of a compound of formulae (I), (II), (III), or (IV) according to the disclosure, nor produce undesirable biological effects or otherwise interact in a deleterious manner with any other component(s) of the pharmaceutical composition.

The pharmaceutical compositions of the disclosure may be prepared by methods know in the pharmaceutical formulation art, for example, see Remington's Pharmaceutical Sciences, 18th Ed., (Mack Publishing Company, Easton, Pa., 1990), which is incorporated herein by reference. In a solid dosage form, a compound of formulae (I), (II), (III), or (IV) according to the disclosure may be admixed with at least one pharmaceutically acceptable excipient such as, for example, sodium citrate or dicalcium phosphate or (a) fillers or extenders, such as, for example, starches, lactose, sucrose, glucose, mannitol, and silicic acid, (b) binders, such as, for example, cellulose derivatives, starch, alginates, gelatin, polyvinylpyrrolidone, sucrose, and gum acacia, (c) humectants, such as, for example, glycerol, (d) disintegrating agents, such as, for example, agar-agar, calcium carbonate, potato or tapioca starch, alginic acid, croscarmellose sodium, complex silicates, and sodium carbonate, (e) solution retarders, such as, for example, paraffin, (f) absorption accelerators, such as, for example, quaternary ammonium compounds, (g) wetting agents, such as, for example, cetyl alcohol, and glycerol monostearate, magnesium stearate and the like, (h) adsorbents, such as, for example, kaolin and bentonite, and (i) lubricants, such as, for example, talc, calcium stearate, magnesium stearate, solid polyethylene glycols, sodium lauryl sulfate, or mixtures thereof. In the case of capsules, tablets, and pills, the dosage forms may also comprise buffering agents. In some embodiments, the excipient is not water. In some embodiments, the excipient is not a solvent (e.g., EtOH, diethyl ether, ethyl acetate, or hydrocarbon-based solvents (e.g., hexanes). In some embodiments, the dosage form is substantially free of water and/or solvents, for example less than about 5% water by mass, less than 2% water by mass, less than 1% water by mass, less than 0.5% water by mass, or less than 0.1% water by mass.

Excipients or pharmaceutically acceptable adjuvants known in the pharmaceutical formulation art may also be used in the pharmaceutical compositions of the disclosure. These include, but are not limited to, preserving, wetting, suspending, sweetening, flavoring, perfuming, emulsifying, and dispensing agents. Prevention of the action of microorganisms may be ensured by inclusion of various antibacterial and antifungal agents, for example, parabens, chlorobutanol, phenol, sorbic acid, and the like. It may also be desirable to include isotonic agents, for example, sugars, sodium chloride, and the like. If desired, a pharmaceutical composition of the disclosure may also contain minor amounts of auxiliary substances such as wetting or emulsifying agents, pH buffering agents, antioxidants, and the like, such as, for example, citric acid, sorbitan monolaurate, triethanolamine oleate, butylated hydroxytoluene, etc.

Solid dosage forms as described above may be prepared with coatings and shells, such as enteric coatings and others well known in the art. They may contain pacifying agents and can also be of such composition that they release the active compound or compounds in a certain part of the intestinal tract in a delayed manner. Non-limiting examples of embedded compositions that may be used are polymeric substances and waxes. The active compounds may also be in microencapsulated form, if appropriate, with one or more of the above-mentioned excipients.

Suspensions, in addition to the active compounds, may contain suspending agents, such as, for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

Solid dosage forms for oral administration, which includes capsules, tablets, pills, powders, and granules, may be used. In such solid dosage forms, the active compound may be mixed with at least one inert, pharmaceutically acceptable excipient (also known as a pharmaceutically acceptable carrier).

Administration of compounds of formulae (I), (II), (III), or (IV) according to the disclosure in pure form or in an appropriate pharmaceutical composition may be carried out via any of the accepted modes of administration or agents for serving similar utilities. Thus, administration may be, for example, orally, buccally, nasally, parenterally (intravenous, intramuscular, or subcutaneous), topically, transdermally, intravaginally, intravesically, or intrasystemically, in the form of solid, semi-solid, lyophilized powder, or liquid dosage forms, such as, for example, tablets, suppositories, pills, soft elastic and hard gelatin capsules, powders, solutions, suspensions, or aerosols, or the like, such as, for example, in unit dosage forms suitable for simple administration of precise dosages. One route of administration may be oral administration, using a convenient daily dosage regimen that can be adjusted according to the degree of severity of the disease-state to be treated.

### EXAMPLES

### Examples

### Example 1: Synthesis of Compounds of Formula (I)

Each of the derivatives exemplified in Table 1 above, where R₄═OH and R₅═H, plus psilocin were constructed in silico using ChemDraw® and Chem3D® 20.1.1. Each structure's energy was minimized using MM2 regression and the final C—N—C exterior angle and interior angle was calculated along with the molecule's overall ground state energy. Results are shown in Table 7.

TABLE 7

| | EXT ANGLE | INT ANGLE | ENERGY |
|---|---|---|---|
| COMPOUND | (DEG) | (DEG) | (KCAL/MOL) |
| Psilocin | 249.9 | 110.1 | 13.36 |
| Azacyclohexyl | 249.5 | 110.5 | 20.69 |
| Azacyclopentyl | 248.4 | 111.6 | 23.38 |
| Azacyclobutyl | 247.0 | 113.0 | 44.83 |
| Azacycpropyl | 241.8 | 118.2 | 125.0 |

Tryptamine analogs with endocyclic amines such as those represented in formula (I), were prepared by synthesis of tryptamine analogs. The following reaction mechanism shows the synthesis of both 3-((1-methylaziridin-2-yl)methyl)-1H-indol-4-ol as well as the acetate prodrug 3-((1-methylaziridin-2-yl)methyl)-1H-indol-4-yl acetate. 4-Acetoxyindole was alkylated at the 3-position to provide the intermediate aldehyde which was converted to the p-toluenesulfonimine by addition of the corresponding sulfonamide. The aziridine moiety was created by the action of in situ generated iodomethyllithium (in this case where R₁ and R₂═H). (Concellón) The N-tosyl bond was cleaved through direct reduction to produce the secondary aziridine which can be alkylated with methyl iodide to produce the target compound (the 4-acetoxy derivative) which was converted to the 4-hydroxy compound through saponification of the following reaction mechanism.

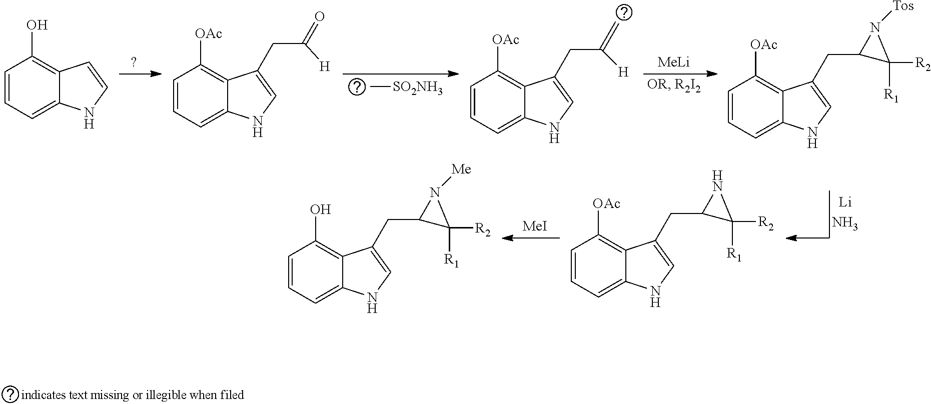

Analytical data for the compounds shown above have been produced via ChemDraw 20.1.1. The spectrum and data for (S)-3-((1-methylaziridin-2-yl)methyl)-1H-indol-4-ol is shown in Table 8.

TABLE 8

| Chemical Formula | C₁₂H₁₄N₂O |
|---|---|
| Exact Mass | 202.11 |
| Molecular Weight | 202.26 |
| M/Z | 202.11 (100.0%), 203.11 (13.8%) |
| Elemental Analysis | C, 71.26; H, 6.98; N, 13.85; O, 7.91 |
| Log P | 1.14 |
| tPSA | 35.27 |
| CLogP | 1.114 |
| pKa | 9.005, 12.265 |

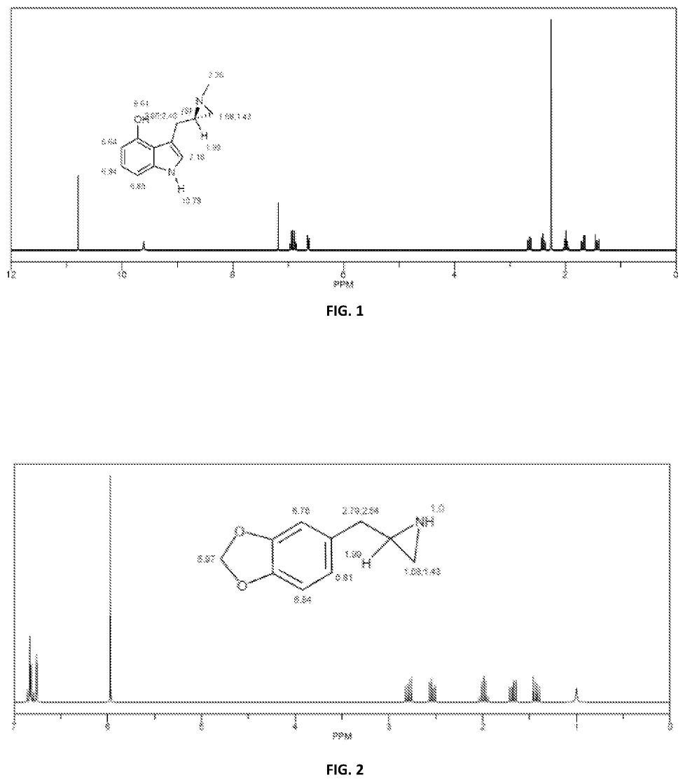

¹H NMR spectrum of (S)-3-((1-methylaziridin-2-yl)methyl)-1H-indol-4-ol is shown in FIG. 1.

### Example 2: Synthesis of Compounds of Formula (II)

Each of the derivatives exemplified in Table 2 above, where R₄═OH and R₅═H, plus psilocin and azacyclobutyl were constructed in silico using ChemDraw® and Chem3D® 20.1.1. Each structure's energy was minimized using MM2 regression and the final C—N—C exterior angle was calculated along with the molecule's overall energy. Results are shown in Table 9.

TABLE 9

| | EXT ANGLE | INT ANGLE | ENERGY |
|---|---|---|---|
| COMPOUND | (DEG) | (DEG) | (KCAL/MOL) |
| Psilocin | 249.9 | 110.1 | 13.36 |
| Azacyclohexyl | 249.2 | 110.8 | 18.55 |
| Azacyclopentyl | 257.8 | 102.2 | 24.33 |
| Azacyclobutyl | 268.2 | 91.8 | 44.35 |
| Aziridinyl | 298.6 | 61.4 | 125.3 |

As exemplified in Table 9, as the ring size decreases (cyclohexyl to aziridinyl), the exterior angle widens and the interior angle closes. These differences may manifest in a different binding affinity for the 5HT receptors.

Tryptamine analogs with exocyclic amines, such as those represented in formula (II), are easily prepared by the venerable Speeter-Anthony synthesis of tryptamine analogs. (Speeter) The following reaction mechanism shows the synthesis of both 3-(2-(azetidin-1-yl)ethyl)-1H-indol-4-yl acetate as well as 3-(2-(azetidin-1-yl)ethyl)-1H-indol-4-ol. Widely available 4-hydroxyindole was acetylated to provide 4-acetoxyindole. Alternatively, 4-acetoxy-indole, since it is also widely available, was purchased as the starting material. 4-Acetoxyindole was acylated at the 3-position with oxalyl chloride to provide the intermediate acyl chloride which was converted to the terminal amide by addition of the cyclic amine, in this case, azacyclobutane. The carbonyls were readily reduced by the action of lithium aluminum hydride to provide the acetoxy tryptamine derivative. This compound can be saponified to provide the 4-hydroxytryptamine derivative as shown in the following reaction mechanism.

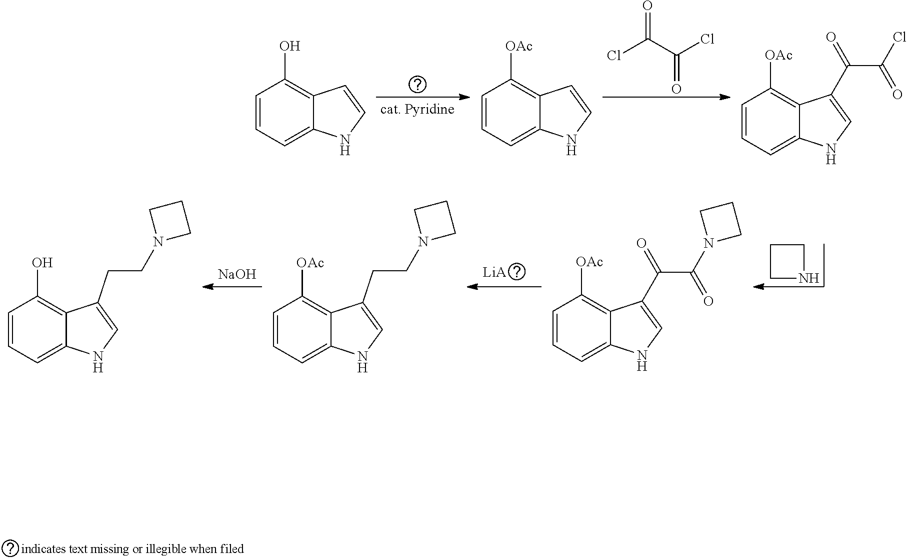

Other compounds of formula (II) were formed in a similar manner.

### Example 3: Synthesis of Compounds of Formula (III)

Commercially available piperonal (shown in the following reaction mechanism) was converted to the vinyl azide in 72% yield in a single pot with first, reaction with ethynyl magnesium bromide to produce the in situ carbinol. A silver-(I) catalyzed hydroazidation yielded the α,β-unsaturated-α-vinyl azide. The a-keto-aziridine was formed by treatment with potassium phosphate (K₃PO₄) in DMF at 95° C. in 86% yield. (Liu). Reduction of the aryl ketone to produce the target compound was accomplished through a Wolff-Kishner reduction shown below or by any number of transformations known to those skilled in the art.

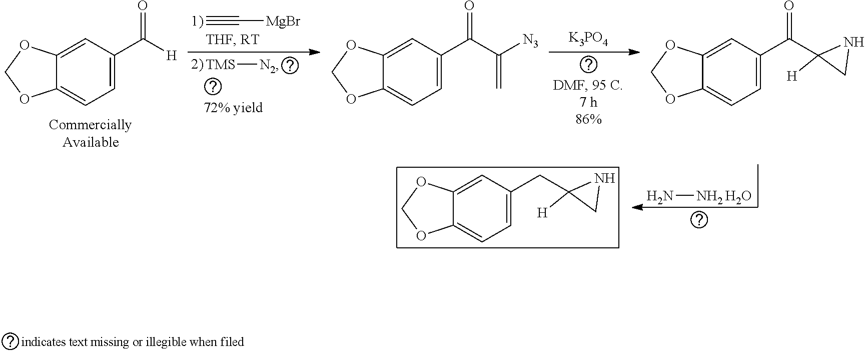

Analytical data for the compounds shown above have been produced via ChemDraw 20.1.1. The spectrum and data for Azacyclopropyl-MDMA analog is shown in Table 10.

TABLE 10

| Chemical Formula | C₁₀H₁₁NO₂ |
|---|---|
| Exact Mass | 177.08 |
| Molecular Weight | 177.20 |
| M/Z | 177.08 (100.0%), 178.08 (11.3%) |
| Elemental Analysis | C, 67.78; H, 6.26; N, 7.90; O, 18.06 |
| Log P | 1.39 |
| tPSA | 4.04 |
| CLogP | 1.31 |
| pKa | 11.573 |

¹H NMR spectrum of Azacyclopropyl-MDMA analog is shown in FIG. 2.

### Example 4: Synthesis of Compounds of Formula (IV)

### Synthesis of 8-methoxy-1,2,3,5,6,11,12,12a-octahydropyrrolo[1′,2′:1,2]azepino[4,5-b]indole (3)

8-methoxy-1,2,3,5,6,11,12,12a-octahydropyrrolo[1′,2′:1,2]azepino[4,5-b]indole (3) is synthesized by the following reaction mechanism.

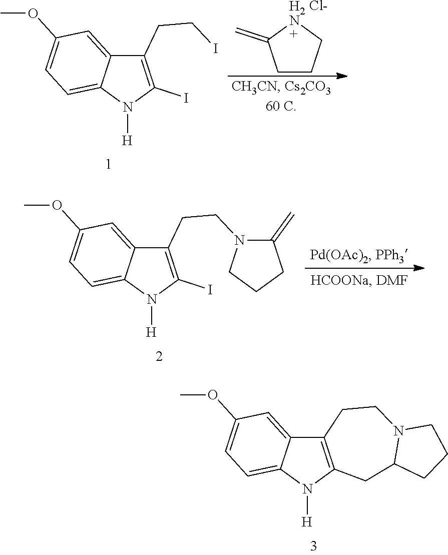

The synthesis of analogue 3 started with 5-Methoxy-2-iodotryptoyl iodide 1 which was prepared by the procedure of Jana and Sinha. Compound 1 was coupled at the alkyl iodide position with 2-methylenepyrrolidine in acetonitrile with cesium carbonate at 60° C. followed by intermolecular Heck type coupling using the analogous method of the same authors to generate compound 3.

### Synthesis of 10-methoxy-1,2,4,5,6,7-hexahydro-3,6-methanoazocino[5,4-b]indole (5)

10-methoxy-1,2,4,5,6,7-hexahydro-3,6-methanoazocino[5,4-b]indole (5) is synthesized by the following reaction mechanism.

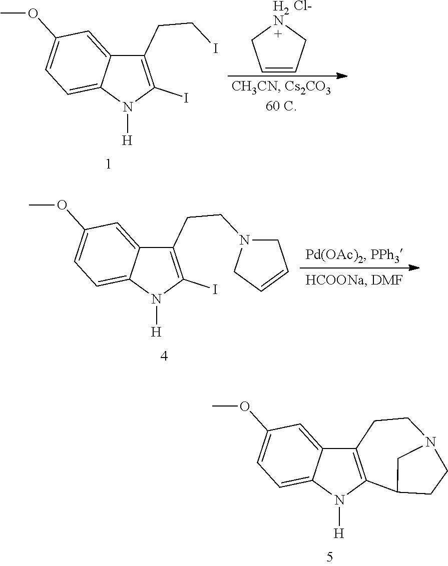

The synthesis of analogue 5 started with 5-Methoxy-2-iodotryptoyl iodide 1 which was prepared by the procedure of Jana and Sinha. Compound 1 was coupled at the alkyl iodide position with 2,5-dihydro-1h-pyrrole in acetonitrile with cesium carbonate at 60° C. followed by intermolecular Heck type coupling using the analogous method of the same authors to generate compound 5.

### REFERENCES

Ballesteros S, Ramon M F, Iturralde M J, Martinez-Arrieta R. (2006). Natural sources of drugs of abuse: magic mushrooms. In: Cole S M, ed. New research on street drugs. New York, 167-186.
Belgers M, Leenaars M, Homberg J R, Ritskes-Hoitinga M, Schellekens A F A, Hooijmans C R. Ibogaine and addiction in the animal model, a systematic review and meta-analysis. Transl Psychiatry. 2016; 6(5):e826. doi:10.1038/tp.2016.71.
Broderick P Phelan F Berger. Ibogaine alters cocaine-induced biogenic amine and psychostimulant dysfunction but not [3H] GBR-12935 binding to the dopamine transporter protein. NIDA Research Monograph Series. 1985; 107:285. https://www.researchgate.net/profile/Kenner_Rice/publication/21719706_The_cannabinoid_re ceptor-pharmacologic_identification_anatomical_localization_and_cloning/links/55229a4d0cf2a2d9e145857b.pdf #page=315.
Broderick P A, Phelan F T, Eng F, Wechsler R T. Ibogaine modulates cocaine responses which are altered due to environmental habituation: In vivo microvoltammetric and behavioral studies. Pharmacol Biochem Behav. 1994; 49(3):711-728. doi:10.1016/0091-3057(94)90092-2.
Brown T K, Alper K. Treatment of opioid use disorder with ibogaine: detoxification and drug use outcomes. Am J Drug Alcohol Abuse. 2018; 44(1):24-36. doi:10.1080/00952990.2017.1320802.
Susanne L. T. Cappendijk, Michailo R. Dzoljic, Inhibitory effects of ibogaine on cocaine self-administration in rats, European Journal of Pharmacology, Volume 241, Issues 2-3, 1993, Pages 261-265.
Carhart-Harris, R. L. & Goodwin, G. M. (2017). Neuropsychopharmacology, 42, 2105-2113.
J. M. Concellón, H. Rodriguez-Solla, C. Simal, Org. Lett., 2008, 10, 4457-4460.
Davis A K, Barsuglia J P, Windham-Herman A-M, Lynch M, Polanco M. Subjective effectiveness of ibogaine treatment for problematic opioid consumption: Short- and long-term outcomes and current psychological functioning. J Psychedelic Stud. 2017; 1(2):65-73. doi:10.1556/2054.01.2017.009.
Dinis-Oliveira, R. J. (2017). Drug Metab. Rev. 49, 84-91.
Dybowski J Landrin. Concerning Iboga, its excitement-producing properties, its composition, and the new alkaloid it contains, ibogaine. CR Acad Sci. 1901; 133:748. https://ibogainedossier.com/dybowski.html.
Dzoljic E D, Kaplan C D, Dzoljic M R. Effect of ibogaine on naloxone-precipitated withdrawal syndrome in chronic morphine-dependent rats. Arch Int Pharmacodyn Ther. 1988; 294:64-70. https://www.ncbi.nlm.nih.gov/pubmed/3233054.
Eivindvik K, Rasmussen K E, Sund R B. (1989). Handling of psilocybin and psilocin by everted sacs of rat jejunum and colon. Acta Pharm Nord 1:295-302.
Glick S D, Rossman K, Steindorf S, Maisonneuve I M, Carlson J N. Effects and aftereffects of ibogaine on morphine self-administration in rats. Eur J Pharmacol. 1991; 195(3):341-345. doi:10.1016/0014-2999(91)90474-5.
Haller A Heckel. Sur l'ibogaine, principe actif d'une plante du genere Tabernaemontana, originarie du Congo. Comptes Rendus de l'Academie des Seances. 1901; 133:850-853.
Horita A, Weber L J. Dephosphorylation of Psilocybin to Psilocin by Alkaline Phosphatase. Proceedings of the Society for Experimental Biology and Medicine. 1961; 106(1):32-34. doi:10.3181/00379727-106-26228.
Jana, G. K.; Sinha, S. Tetrahedron 68, 2012, 7155-7165 and supplementary information within.
Johnson, M. W. & Griffiths, R. R. (2017). Neurotherapeutics 14, 734-740.
Alan R. Katritzky, et al, Structure of Small and Large Rings, Handbook of Heterocyclic Chemistry (Third Edition), 2010, 2.5.4.2.1.2 Pyramidal inversion at ring nitrogen.
Adam K. Klein, Muhammad Chatha, Lauren J. Laskowski, Emilie I. Anderson, Simon D. Brandt, Stephen J. Chapman, John D. McCorvy, and Adam L. Halberstadt, Investigation of the Structure-Activity Relationships of Psilocybin Analogues, ACS Pharmacology & Translational Science 2021 4 (2), 533-542, DOI: 10.1021/acsptsci.0c00176.
Liu, Z., Liu, J., Zhang, L., Liao, P., Song, J., & Bi, X. (2014). Silver(I)-Catalyzed Hydroazidation of Ethynyl Carbinols: Synthesis of 2-Azidoallyl Alcohols. Angewandte Chemie International Edition, 53(21), 5305-5309. doi:10.1002/anie.201310264.
Maisonneuve I M, Keller R W Jr, Glick S D. Interactions between ibogaine, a potential anti-addictive agent, and morphine: an in vivo microdialysis study. Eur J Pharmacol. 1991; 199(1):35-42. doi:10.1016/0014-2999(91)90634-3.
Mash D C, Duque L, Page B, Allen-Ferdinand K. Ibogaine Detoxification Transitions Opioid and Cocaine Abusers Between Dependence and Abstinence: Clinical Observations and Treatment Outcomes. Front Pharmacol. 2018; 9:529. doi:10.3389/fphar.2018.00529.
D. E. Nichols, Structure-activity relationships of serotonin 5-HT2A agonists, Wiley Interdisciplinary Reviews: Membrane Transport and Signaling, 2012, 1(5), 559-579.
Noller G E, Frampton C M, Yazar-Klosinski B. Ibogaine treatment outcomes for opioid dependence from a twelve-month follow-up observational study. Am J Drug Alcohol Abuse. 2018; 44(1):37-46. doi:10.1080/00952990.2017.1310218.
A. Padwa, A. D. Woolhouse, Aziridines, Azirines and Fused-ring Derivatives, Comprehensive Heterocyclic Chemistry, 1984, 5.04.2.7 Nitrogen Inversion.
Passie T, Seifert J, Schneider U, Emrich H M. The pharmacology of psilocybin. Addict Biol. 2002 October; 7(4):357-64. doi: 10.1080/1355621021000005937. PMID: 14578010.
Schenberg E E, de Castro Comis M A, Chaves B R, da Silveira D X. Treating drug dependence with the aid of ibogaine: a retrospective study. J Psychopharmacol. 2014; 28(11):993-1000. doi:10.1177/0269881114552713.
Sershen H, Hashim A, Lajtha A. Ibogaine reduces preference for cocaine consumption in C57BL/6 By mice. Pharmacol Biochem Behav. 1994; 47(1):13-19. doi:10.1016/0091-3057(94)90105-8.
M. E. Speeter and W. C. Anthony, J. Am. Chem. Soc., 1954, 76, 6208-6212.
Winkelman M. Psychedelics as medicines for substance abuse rehabilitation: evaluating treatments with LSD, Peyote, Ibogaine and Ayahuasca. Curr Drug Abuse Rev. 2014; 7(2):101-116. doi:10.2174/1874473708666150107120011.

What is claimed is:

1. A compound of formula (I): 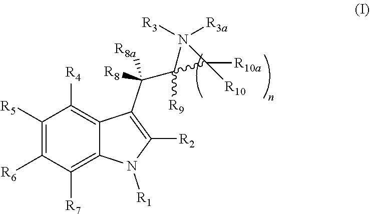
wherein:
R₁ is selected from hydrogen, optionally substituted C₁-C₆ alkyl, optionally substituted C₂-C₆ alkenyl, optionally substituted aryl, —OR₁₁, —C(O)R₁₁, —C(O)OR₁₁, —SO₂R₁₁, and —C(O)NR₁₁R₁₂;
R₂, R₄, R₅, R₆, R₇, R₈, R₈ₐ, R₉, R₁₀, and R₁₀ₐ are for each occurrence independently selected from hydrogen, optionally substituted C₁-C₆ alkyl, optionally substituted C₂-C₆ alkenyl, optionally substituted C₁-C₆-heteroalkyl, optionally substituted heteroaryl, optionally substituted aryl, halogen, hydroxy, CN, —OR₁₁, —OC(O)R₁₁, —OC(O)OR₁₁, —OSO₂R₁₁, —OC(O)NR₁₁R₁₂, —OP(O)(OH)₂, and —OP(O)(OH)OP(O)(OH)₂;
R₃ is selected from hydrogen, optionally substituted C₁-C₆ alkyl, optionally substituted C₂-C₆ alkenyl, optionally substituted aryl, and —SO₂R₁₁;
R₃ₐ is selected from an electron pair, hydrogen, optionally substituted C₁-C₆ alkyl, optionally substituted C₂-C₆ alkenyl, and optionally substituted aryl, wherein when R₃ₐ is not an electron pair the compound of formula (I) is a quaternary amine cation with a pharmaceutically acceptable anion, X⁻;
R₁₁ and R₁₂ are for each occurrence independently selected from hydrogen, optionally substituted C₁-C₆ alkyl, optionally substituted C₂-C₆ alkenyl, and optionally substituted aryl; and
n is an integer defining the variable number of ring carbons carrying R₁₀ and R₁₀ₐ and is selected from 1 to 4.

2. The compound of claim 1, wherein at least one of R₄ or R₅ is selected from hydroxy, —OR₁₁, —OC(O)R₁₁, —OC(O)OR₁₁, —OSO₂R₁₁, —OC(O)NR₁₁R₁₂, —OP(O)(OH)₂, and —OP(O)(OH)OP(O)(OH)₂.

3. The compound of claim 2, wherein at least one of R₄ or R₅ is selected from hydroxy and —OC(O)R₁₁.

4. The compound of claim 3, wherein R₁₁ is an optionally substituted C₁-C₆ alkyl.

5. The compound of claim 4, wherein R₁₁ is an unsubstituted C₁-C₆ alkyl.

6. The compound of claim 5, wherein R₁₁ is methyl.

7. The compound of any of claims 1-6, wherein R₃ is selected from optionally substituted C₁-C₆ alkyl and optionally substituted C₂-C₆ alkenyl.

8. The compound of claim 7, wherein R₃ is an unsubstituted C₁-C₆ alkyl.

9. The compound of claim 8, wherein R₃ is methyl.

10. The compound of any of claims 1-9, wherein R₃ₐ is an electron pair.

11. The compound of any of claims 1-10, wherein R₆ is hydrogen.

12. The compound of any of claims 1-11, wherein R₇ is hydrogen.

13. The compound of any of claims 1-12, wherein R₉ is hydrogen.

14. The compound of any of claims 1-13, wherein R₁ is selected from hydrogen and optionally substituted C₁-C₆ alkyl.

15. The compound of any of claims 1-14, wherein R₁ is a 2-(S)-methyl-2-N,N-dimethylamino-ethyl or 2-(R)-methyl-2-N,N-dimethylamino-ethyl.

16. The compound of any of claims 1-15, wherein R₁₀ is hydrogen.

17. The compound of any of claims 1-16, wherein R₁₀, is hydrogen.

18. The compound of any of claims 1-17, wherein R₈ is hydrogen.

19. The compound of any of claims 1-18, wherein R₈ₐ is hydrogen.

20. The compound of any of claims 1-19, wherein n=1.

21. The compound of any claims 1-19, wherein n=2.

22. The compound of any of claims 1-19, wherein n=3.

23. The compound of any of claims 1-19, wherein n=4.

24. The compound of any of claims 1-23, wherein R₂ is hydrogen.

25. A compound of formula (II): 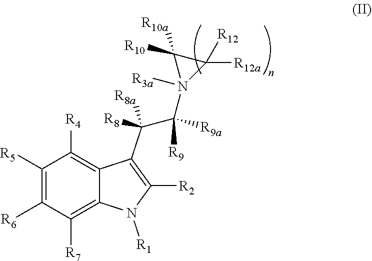
wherein:
R₁ is selected from hydrogen, optionally substituted C₁-C₆ alkyl, optionally substituted C₂-C₆ alkenyl, optionally substituted aryl, —OR₁₁, —C(O)R₁₁, —C(O)OR₁₁, —SO₂R₁₁, and —C(O)NR₁₁R₁₃;
R₂, R₄, R₅, R₆, R₇, R₈, R₈ₐ, R₉, R₉ₐ, R₁₀, R₁₀ₐ, R₁₂, and R₁₂ₐ are for each occurrence independently selected from hydrogen, optionally substituted C₁-C₆ alkyl, optionally substituted C₂-C₆ alkenyl, optionally substituted C₁-C₆-heteroalkyl, optionally substituted heteroaryl, optionally substituted aryl, halogen, hydroxy, CN, —OR₁₁, —OC(O)R₁₁, —OC(O)OR₁₁, —OSO₂R₁₁, —OC(O)N R₁₁R₁₃, —OP(O)(OH)₂, and —OP(O)(OH)OP(O)(OH)₂;
R₃ₐ is selected from an electron pair, hydrogen, optionally substituted C₁-C₆ alkyl, optionally substituted C₂-C₆ alkenyl, and optionally substituted aryl, wherein when R₃ₐ is not an electron pair the compound of formula (II) is a quaternary amine cation with a pharmaceutically acceptable anion, X⁻;
R₁₁ and R₁₃ are for each occurrence independently selected from hydrogen, optionally substituted C₁-C₆ alkyl, optionally substituted C₂-C₆ alkenyl, and optionally substituted aryl; and
n is an integer defining the variable number of ring carbons carrying R₁₂ and R₁₂ₐ and is selected from 1 to 4.

26. The compound of claim 25, wherein at least one of R₄ or R₅ is selected from hydroxy, —OR₁₁, —OC(O)R₁₁, —OC(O)OR₁₁, —OSO₂R₁₁, —OC(O)NR₁₁R₁₂, —OP(O)(OH)₂, and —OP(O)(OH)OP(O)(OH)₂.

27. The compound of claim 26, wherein at least one of R₄ or R₅ is selected from hydroxy and —OC(O)R₁₁.

28. The compound of claim 27, wherein R₁₁ is an optionally substituted C₁-C₆ alkyl.

29. The compound of claim 28, wherein R₁₁ is an unsubstituted C₁-C₆ alkyl.

30. The compound of claim 29, wherein R₁₁ is methyl.

31. The compound of any of claims 25-30, wherein R₃ₐ is an electron pair.

32. The compound of any of claims 25-31, wherein R₆ is hydrogen.

33. The compound of any of claims 25-32, wherein R₇ is hydrogen.

34. The compound of any of claims 25-33, wherein R₉ is hydrogen.

35. The compound of any of claims 25-34, wherein R₉ₐ is hydrogen.

36. The compound of any of claims 25-35, wherein R₈ is hydrogen.

37. The compound of any of claims 25-36, wherein R₈ₐ is hydrogen.

38. The compound of any of claims 25-37, wherein R₁ is selected from hydrogen and optionally substituted C₁-C₆ alkyl.

39. The compound of any of claims 25-38, wherein R₁ is selected from 2-(S)-methyl-2-N,N-dimethylamino-ethyl or 2-(R)-methyl-2-N,N-dimethylamino-ethyl.

40. The compound of any of claims 25-39, wherein R₁₀ is hydrogen.

41. The compound of any of claims 25-40, wherein R₁₀ₐ is hydrogen.

42. The compound of any of claims 25-41, wherein R₁₂ is hydrogen.

43. The compound of any of claims 25-42, wherein R₁₂ₐ is hydrogen.

44. The compound of any of claims 25-43, wherein n=1.

45. The compound of any of claims 25-43, wherein n=2.

46. The compound of any of claims 25-43, wherein n=3.

47. The compound of any of claims 25-43, wherein n=4.

48. The compound of claim 45, wherein when n=2, R₃ₐ is not an electron pair and/or at least one of R₈, R₈ₐ, R₉, or R₉ₐ is not hydrogen.

49. A compound of formula (III): 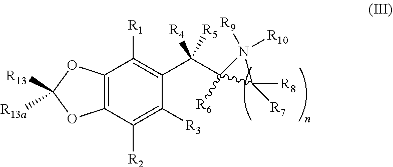
wherein:
R₁, R₂, and R₃ are independently selected from hydrogen, optionally substituted C₁-C₆ alkyl, optionally substituted C₂-C₆ alkenyl, optionally substituted aryl, halogen, —OR₁₁, —C(O)R₁₁, —C(O)OR₁₁, —OC(O)R₁₁, C(O)NR₁₁R₁₂, CN, C(NR₁₂)R₁₁, C(NOR₁₂)OR₁₁, —SO₂R₁₁, and —C(O)NR₁₁R₁₂;
R₄, R₅, R₆, R₇, and R₈ are for each occurrence independently selected from hydrogen, optionally substituted C₁-C₆ alkyl, optionally substituted C₂-C₆ alkenyl, optionally substituted C₁-C₆ heteroalkyl, optionally substituted heteroaryl, optionally substituted aryl, halogen, hydroxy, —OR₁₁, —OC(O)R₁₁, —OC(O)OR₁₁, OSO₂R₁₁, and —OC(O)NR₁₁R₁₂;
R₉, is selected from hydrogen, optionally substituted C₁-C₆ alkyl, optionally substituted C₂-C₆ alkenyl, optionally substituted aryl, and —SO₂R₁₁;
R₁₀ is selected from an electron pair, hydrogen, optionally substituted C₁-C₆ alkyl, optionally substituted C₂-C₆ alkenyl, and optionally substituted aryl, wherein when R₁₀ is not an electron pair the compound of formula (III) is a quaternary amine cation with a pharmaceutically acceptable anion, X⁻;
R₁₁ and R₁₂ are for each occurrence independently selected from hydrogen, optionally substituted C₁-C₆ alkyl, optionally substituted C₂-C₆ alkenyl, and optionally substituted aryl;
R₁₃ and R₁₃ₐ are for each occurrence independently selected from hydrogen, optionally substituted C₁-C₆ alkyl, optionally substituted C₂-C₆ alkenyl, and optionally substituted aryl, or R₁₃ and R₁₃ₐ are taken together with the carbon to which they are bound to form a 3- to 6-membered cycloalkyl; and
n is an integer defining the variable number of ring carbons carrying R₇ and R₈ and is selected from 1 to 4.

50. The compound of claim 49, wherein R₁ is hydrogen.

51. The compound of claim 49 or claim 50, wherein R₂ is hydrogen.

52. The compound of any of claims 49-51, wherein R₃ is hydrogen.

53. The compound of any of claims 49-52, wherein R₄ is hydrogen.

54. The compound of any of claims 49-53, wherein R₅ is hydrogen.

55. The compound of any of claims 49-54, wherein R₆ is hydrogen.

56. The compound of any of claims 49-55, wherein R₉ is an optionally substituted C₁-C₆ alkyl.

57. The compound of claim 56, wherein R₉ is an unsubstituted C₁-C₆ alkyl.

58. The compound of claim 57, wherein R₉ is methyl.

59. The compound of any of claims 49-58, wherein R₁₀ is an electron pair.

60. The compound of any of claims 49-59, wherein R₇ is hydrogen.

61. The compound of any of claims 49-60, wherein R₈ is hydrogen.

62. The compound of any of claims 49-61, wherein R₁₃ is hydrogen.

63. The compound of any of claims 49-62, wherein R₁₃ₐ is hydrogen.

64. The compound of any of claims 49-61, wherein R₁₃ and R₁₃ₐ are taken together with the carbon to which they are bound to form a cyclopropyl group.

65. The compound of any of claims 49-61, wherein R₁₃ and R₁₃ₐ are taken together with the carbon to which they are bound to form a cyclobutyl group.

66. The compound of any of claims 49-61, wherein R₁₃ and R₁₃ₐ are taken together with the carbon to which they are bound to form a cyclopentyl group.

67. The compound of any of claims 49-61, wherein R₁₃ and R₁₃ₐ are taken together with the carbon to which they are bound to form a cyclohexyl group.

68. The compound of any of claims 49-67, wherein n=1.

69. The compound of any of claims 49-67, wherein n=2.

70. The compound of any of claims 49-67, wherein n=3.

71. The compound of any of claims 49-67, wherein n=4.

72. A compound of formula (IV): 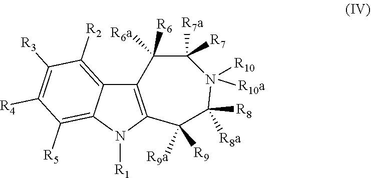
wherein:
R₁ is selected from hydrogen, optionally substituted C₁-C₆ alkyl, optionally substituted C₂-C₆ alkenyl, optionally substituted aryl, —OR₁₁, —C(O)R₁₁, —C(O)OR₁₁, —SO₂R₁₁, and —C(O)N R₁₁R₁₂;
R₂, R₃, R₄, R₅, R₆, R₆ₐ, R₇, R₇ₐ, R₈, R₈ₐ, R₉, and R₉ₐ are for each occurrence independently selected from hydrogen, optionally substituted C₁-C₆ alkyl, optionally substituted C₂-C₆ alkenyl, optionally substituted C₁-C₆-heteroalkyl, optionally substituted heteroaryl, optionally substituted aryl, halogen, hydroxy, —OR₁₁, —OC(O)R₁₁, —OC(O)OR₁₁, —OSO₂R₁₁, and —OC(O)NR₁₁R₁₂;
R₁₀ is selected from an electron pair, hydrogen, optionally substituted C₁-C₆ alkyl, optionally substituted C₂-C₆ alkenyl, and optionally substituted aryl, wherein when R₁₀ is not an electron pair the compound of formula (IV) is a quaternary amine cation with a pharmaceutically acceptable anion, X⁻;
R₁₀ₐ is taken together with one of R₈, R₈ₐ, R₉, or R₉ₐ to form optionally substituted 3- to 6-membered heterocyclic ring; and
R₁₁ and R₁₂ are for each occurrence independently selected from hydrogen, optionally substituted C₁-C₆ alkyl, optionally substituted C₂-C₆ alkenyl, and optionally substituted aryl.

73. The compound of claim 72, wherein at least one of R₂, R₃, R₄, or R₅ is selected from hydroxy, —OR₁₁, —OC(O)R₁₁, —OC(O)OR₁₁, —OSO₂R₁₁, and —OC(O)NR₁₁R₁₂.

74. The compound of claim 73, wherein at least one of R₂, R₃, R₄, or R₅ is selected from hydroxy and —OR₁₁.

75. The compound of claim 74, wherein at least one of R₂, R₃, R₄, or R₅ is —OR₁₁.

76. The compound of claim 75, wherein R₁₁ is unsubstituted C₁-C₆ alkyl.

77. The compound of claim 76, wherein R₁₁ is methyl.

78. The compound of any of claims 72-77, wherein R₃ is methoxy.

79. The compound of any of claims 72-78, wherein R₁₀ₐ, is taken together with R₈ to form an optionally substituted 3-membered heterocyclic ring.

80. The compound of any of claims 72-78, wherein R₁₀ₐ, is taken together with R₈ to form an optionally substituted 4-membered heterocyclic ring.

81. The compound of any of claims 72-78, wherein R₁₀ₐ, is taken together with R₈ to form an optionally substituted 5-membered heterocyclic ring.

82. The compound of any of claims 72-78, wherein R₁₀ₐ, is taken together with R₈ to form an optionally substituted 6-membered heterocyclic ring.

83. The compound of any of claims 72-78, wherein R₁₀ₐ, is taken together with R₉ to form an optionally substituted 4-membered heterocyclic ring.

84. The compound of any of claims 72-78, wherein R₁₀ₐ, is taken together with R₉ to form an optionally substituted 5-membered heterocyclic ring.

85. The compound of any of claims 72-78, wherein R₁₀ₐ, is taken together with R₉ to form an optionally substituted 6-membered heterocyclic ring.

86. The compound of any of claims 72-85, wherein the heterocyclic ring is unsubstituted.

87. The compound of any of claims 72-86, wherein R₁₀ is a lone pair.

88. The compound of any of claims 72-87, wherein R₆ is hydrogen.

89. The compound of any of claims 72-88, wherein R₆ₐ is hydrogen.

90. The compound of any of claims 72-89, wherein R₇ is hydrogen.

91. The compound of any of claims 72-90, wherein R₇ₐ is hydrogen.

92. The compound of any of claims 72-91, wherein R₁ is hydrogen.

93. A composition comprising, consisting essentially of, or consisting of a compound according to any one of claims 1-92 and an excipient.

94. A pharmaceutical composition comprising, consisting essentially of, or consisting of a therapeutically effective amount of a compound according to any one of claims 1-92 and a pharmaceutically acceptable excipient.

95. A composition comprising, consisting essentially of, or consisting of as a first active component: a compound according to any one of claims 1-92; and as a second active component selected from (a) a serotonergic drug, (b) a purified psilocybin derivative, (c) one or two purified cannabinoids, and (d) a purified terpene; and a pharmaceutically acceptable excipient.

96. A composition comprising, consisting essentially of, or consisting of as a first active component: a compound according to any one of claims 1-92; and a second active component comprising an adrenergic drug or a dopaminergic drug; and a pharmaceutically acceptable excipient.

97. A composition comprising, consisting essentially of, or consisting of as a first active component: a compound according to any one of claims 1-92; and a second active component comprising a purified monoamine oxidase inhibitor; and a pharmaceutically acceptable excipient.

98. A composition comprising, consisting essentially of, or consisting of as a first active component: a compound according to any one of claims 1-92; and a second active component comprising a purified erinacine or a purified hericenone; and a pharmaceutically acceptable excipient.

99. A method of preventing or treating a psychological disorder comprising the steps of:
identifying a subject in need of treatment or prevention; and
administering to the subject in need thereof a therapeutically effective amount of a compound according to any one of claims 1-92 or a composition according to any one of claims 93-98.

100. A method of preventing or treating inflammation and/or pain comprising the steps of:
identifying a subject in need of treatment or prevention; and
administering to the subject in need thereof a therapeutically effective amount of a compound according to any one of claims 1-92 or a composition according to any one of claims 93-98.

101. A method of preventing or treating sexual health disorders comprising the steps of:
identifying a subject in need of treatment or prevention; and
administering to the subject in need thereof a therapeutically effective amount of a compound according to any one of claims 1-92 or a composition according to any one of claims 93-98.

102. A method of preventing or treating women's health disorders comprising the steps of:
identifying a subject in need of treatment or prevention; and
administering to the subject in need thereof a therapeutically effective amount of a compound according to any one of claims 1-92 or a composition according to any one of claims 93-98.

103. A method of modulating activity of a mitogen-activated protein kinase (MAPK), comprising administering a MAPK activity modulator composition comprising a compound according to any one of claims 1-92 or a composition according to any one of claims 93-98.

104. A method of modulating neurogenesis, comprising administering a neurogenesis modulator composition comprising a compound according to any one of claims 1-92 or a composition according to any one of claims 93-98.

105. A method of modulating neurite outgrowth, comprising administering a MAPK activity modulator composition comprising a compound according to any one of claims 1-92 or a composition according to any one of claims 93-98.

106. A method of treating and/or preventing a substance use disorder comprising the steps of:
identifying a subject in need of treatment or prevention; and
administering to the subject in need thereof a therapeutically effective amount of a compound according to any one of claims 1-92 or a composition according to any one of claims 93-98.

107. The method of claim 106, wherein the substance use disorder is selected from the group consisting of opioid use disorder; cannabis or marijuana use disorder; nicotine use disorder; stimulant use disorder; sedative use disorder; hypnotic use disorder; anxiolytic use disorder; hallucinogen use disorder; phencyclidine use disorder; inhalant use disorder; caffeine use disorder; and alcohol use disorder.
